(19) United States
(12) Patent Application Publication
(10) Pub. No.: US 2009/0232801 A1
(43) Pub. Date: Sep. 17, 2009

(54) Humanized Antibodies Which Bind To AB (1-42) Globulomer And Uses Thereof

(75) Inventors: Heinz Hillen, Hassloch (DE); Stefan Barghorn, Mannheim (DE); Ulrich Ebert, Mannheim (DE); Andreas R. Striebinger, Speyer (DE); Patrick Keller, Darmstadt (DE); Boris Labkovsky, Wales, MA (US); Paul R. Hinton, Sunnyvale, CA (US); Veronica M. Juan, Menlo Park, CA (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOT LABORATORIES, ABBOTT PARK, IL (US)

(21) Appl. No.: 12/129,395

(22) Filed: May 29, 2008

Related U.S. Application Data
(60) Provisional application No. 60/940,931, filed on May 30, 2007.

(51) Int. Cl. A61K 39/395; G01N 33/53; C12P 21/04; C12N 5/10; C12N 1/21; C12N 1/15; C12N 1/19; C12N 15/63; C07K 16/00; C07H 21/04

(52) U.S. Cl. 424/133.1; 424/139.1; 424/141.1; 435/7.1; 435/69.6; 435/69.7; 435/325; 435/348; 435/349; 435/358; 435/420; 435/252.33; 435/254.11; 435/254.21; 435/320.1; 530/387.3; 530/387.9; 530/388.1; 530/389.1; 530/391.1; 530/391.3; 530/391.7; 536/23.4; 536/23.53

(57) ABSTRACT

The present invention relates to binding proteins and, in particular, humanized antibodies that may be used, for example, in the diagnosis, treatment and prevention of Alzheimer's Disease and related conditions.

The subject application claims priority to U.S. provisional patent application No. 60/940,931, filed on May 30, 2007, hereby incorporated in its entirety by reference.

### CROSS-REFERENCE TO RELATED APPLICATIONS

The subject application relates to International Appln. No. PCT/EP2006/011530 filed on Nov. 30, 2006.

### REFERENCE TO JOINT RESEARCH AGREEMENT

Contents of this application are under a joint research agreement entered into by and between Protein Design Labs, Inc. and Abbott Laboratories on Aug. 31, 2006, and directed to humanized amyloid beta antibodies.

### BACKGROUND OF THE INVENTION

1. Field of the Invention

The present invention relates to antibodies that may be used, for example, in the diagnosis, treatment and prevention of conditions such as amyloidoses (e.g., Alzheimer's Disease) and related conditions.

2. Background Information

Alzheimer's Disease (AD) is a neurodegenerative disorder characterized by a progressive loss of cognitive abilities and by characteristic neuropathological features comprising amyloid deposits, neurofibrillary tangles and neuronal loss in several regions of the brain (see Hardy and Selkoe (Science 297, 353 (2002); Mattson Nature 431, 7004 (2004). The principal constituents of amyloid deposits are amyloid beta-peptides (Aβ), with the 42 amino acid-long type (Aβ1-42) being the most prominent.

In particular, amyloid β(1-42) protein is a polypeptide having 42 amino acids which is derived from the amyloid precursor protein (APP) by proteolytic processing. This also includes, in addition to human variants, isoforms of the amyloid β(1-42) protein present in organisms other than humans, in particular, other mammals, especially rats. This protein, which tends to polymerize in an aqueous environment, may be present in very different molecular forms.

A simple correlation of the deposition of insoluble protein with the occurrence or progression of dementia disorders such as, for example, Alzheimer's disease, has proved to be unconvincing (Terry et al., Ann. Neurol. 30. 572-580 (1991); Dickson et al., Neurobiol. Aging 16, 285-298 (1995)). In contrast, the loss of synapses and cognitive perception seems to correlate better with soluble forms of Aβ(1-42)(Lue et al., Am. J. Pathol. 155, 853-862 (1999); McLean et al., Ann. Neurol. 46, 860-866 (1999)).

Although polyclonal and monoclonal antibodies have been raised in the past against Aβ(1-42), none have proven to produce the desired therapeutic effect without also causing serious side effects in animals and/or humans. For example, passive immunization results from preclinical studies in very old APP23 mice which received a N-terminal directed anti-Aβ(1-42) antibody once weekly for 5 months indicate therapeutically relevant side effects. In particular, these mice showed an increase in number and severity of microhemorrhages compared to saline-treated mice (Pfeifer et al., Science 2002 298:1379). A similar increase in hemorrhages was also described for very old (>24 months) Tg2576 and PDAPP mice (Wilcock et al., J Neuroscience 2003, 23: 3745-51; Racke et al., J Neuroscience 2005, 25:629-636). In both strains, injection of anti-Aβ(1-42) resulted in a significant increase of microhemorrhages. Thus, a tremendous, unmet therapeutic need exists for the development of biologics that prevent or slow down the progression of the disease without inducing negative and potentially lethal effects on the human body. Such a need is particularly evident in view of the increasing longevity of the general population and, with this increase, an associated rise in the number of patents annually diagnosed with Alzheimer's Disease or related disorders. Further, such antibodies will allow for proper diagnosis of Alzheimer's Disease in a patient experiencing symptoms thereof, a diagnosis which can only be confirmed upon autopsy at the present time. Additionally, the antibodies will allow for the elucidation of the biological properties of the proteins and other biological factors responsible for this debilitating disease.

All patents and publications referred to herein are hereby incorporated in their entirety by reference.

### SUMMARY OF THE INVENTION

The present invention pertains to binding proteins, particularly antibodies (such as the one referred to herein by the terms “8F5” for the mouse/murine monoclonal antibody and “8F5 hum8” for the humanized 8F5) capable of binding to soluble oligomers and, in particular, Aβ(1-42) globulomer present in the brain of a patient having Alzheimer's Disease. For purposes herein, these binding proteins and, in particular, antibodies will be referred to as “globulomer-epitope specific antibodies”. This means that the antibodies bind to one or more epitopes (e.g., the (1-42) amino acid region of the Aβ(1-42) peptide) of the globulomer or antigen thought to be the cause of Alzheimer's disease. Further, the present invention also provides methods of producing and using these binding proteins or portions thereof.

One aspect of this invention pertains to a binding protein (e.g., antibody) comprising an antigen binding domain capable of binding to an Aβ(1-42) globulomer. In one embodiment, the antigen-binding domain comprises at least one CDR comprising an amino acid sequence selected from the group consisting of:
CDR-VH1. X₁-X₂-X₃-X₄-X₅ (SEQ ID NO:5), wherein:
   X₁ is S;
   X₂ is Y;
   X₃ is G;
   X₄ is M; and
   X₅ is S.
CDR-VH2. X₁-X₂-X₃-X₄-X₅-X₆-X₇-X₈-X₉-X₁₀-X₁₁-X₁₂-X₁₃-X₁₄-X₁₅-X₁₆-X₁₇ (SEQ ID NO:6), wherein:
   X₁ is S;
   X₂ is I;
   X₃ is N;
   X₄ is S;
   X₅ is N;
   X₆ is G;
   X₇ is G;
   X₈ is S;
   X₉ is T;
   X₁₀ is Y;
   X₁₁ is Y;
   X₁₂ is P;
   X₁₃ is D;
   X₁₄ is S;
   X₁₅ is V;
   X₁₆ is K; and
   X₁₇ is G.
CDR-VH3. X₁-X₂-X₃-X₄ (SEQ ID NO:7), wherein:
   X₁ is S;
   X₂ is G;
   X₃ is D; and
   X₄ is Y.
CDR-VL1. X₁-X₂-X₃-X₄-X₅-X₆-X₇-X₈-X₉-X₁₀-X₁₁-X₁₂-X₁₃-X₁₄-X₁₅-X₁₆ (SEQ ID NO:8), wherein:
   X₁ is R;
   X₂ is S;
   X₃ is S;
   X₄ is Q;
   X₅ is S;
   X₆ is L;
   X₇ is V;
   X₈ is Y;
   X₉ is S;
   X₁₀ is N;
   X₁₁ is G;
   X₁₂ is D;
   X₁₃ is T;
   X₁₄ is Y;
   X₁₅ is L; and
   X₁₆ is H.
CDR-VL2. X₁-X₂-X₃-X₄-X₅-X₆-X₇ (SEQ ID NO: 9) wherein;
   X₁ is K;
   X₂ is V;
   X₃ is S;
   X₄ is N;
   X₅ is R;
   X₆ is F; and
   X₇ is S.
and
CDR-VL3. X₁-X₂-X₃-X₄-X₅-X₆-X₇-X₈-X₉ (SEQ ID NO:10) wherein:
   X₁ is S;
   X₂ is Q;
   X₃ is S;
   X₄ is T;
   X₅ is H;
   X₆ is V;
   X₇ is P;
   X₈ is W; and
   X₉ is T.

Preferably, the antigen binding domain comprises at least one CDR comprising an amino acid sequence selected from the group consisting of residues 31-35 (i.e., SYGMS (SEQ ID NO:11); 8F5 VH CDR1) of SEQ ID NO.:1; residues 50-66 (i.e., SINSNGGSTYYPDSVKG (SEQ ID NO:12); 8F5 VH CDR2) of SEQ ID NO.:1; residues 98-108 (i.e., SGDY (SEQ ID NO:13); 8F5 VH CDR3) of SEQ ID NO.:1; residues 24-39 (i.e., RSSQSLVYSNGDTYLH (SEQ ID NO:14); 8F5 VL CDR1) of SEQ ID NO.:2; residues 55-61 (i.e., KVSNRFS (SEQ ID NO:15); 8F5 VL CDR2) of SEQ ID NO.:2; and residues 94-102 (i.e., SQSTHVPWT (SEQ ID NO:16); 8F5 VL CDR3) of SEQ ID NO.:2. In a preferred embodiment, the binding protein comprises at least 3 CDRs selected from the group consisting of the sequences disclosed above. More preferably, the 3 CDRs selected are from sets of variable domain CDRs selected from the group consisting of:

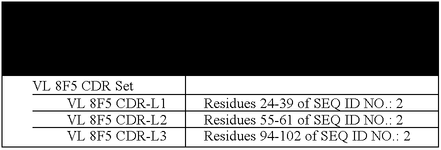

In one embodiment, the binding protein of the invention comprises at least two variable domain CDR sets.

More preferably, the two variable domain CDR sets are: VH 8F5 CDR Set & VL 8F5 CDR Set. In another embodiment the binding protein disclosed above further comprises a human acceptor framework. Preferably the human acceptor framework comprises an amino acid sequence selected from the group consisting of:

| | |
|---|---|
| EVQLLESGGGLVQPGGSLRLSCAASGFTFS; | (SEQ ID NO:17) |
| WVRQAPGKGLEWVS; | (SEQ ID NO:18) |
| RFTISRDNSKNTLYLQMNSLRAEDTAVYYCA; | (SEQ ID NO:19) |
| WGQGTLVTVSS; | (SEQ ID NO:20) |
| DIVMTQSPLSLPVTPGEPASISC; | (SEQ ID NO:21) |
| WYLQKPGQSPQLLIY; | (SEQ ID NO:22) |
| GVPDRFSGSGSGTDFTLKISRVEAEDVGVYYC; | (SEQ ID NO:23) |
| and | |
| FGGGTKVEIKR. | (SEQ ID NO:24) |

In a preferred embodiment, the binding protein is a CDR grafted antibody or antigen binding portion thereof capable of binding to an Aβ(1-42) globulomer. Preferably, the CDR grafted antibody or antigen binding portion thereof comprises one or more CDRs disclosed above. More preferably, the CDR grafted antibody or antigen binding portion thereof comprises at least one variable domain having an amino acid sequence selected from the group consisting of SEQ ID NO:25 and SEQ ID NO:26. Most preferably, the CDR grafted antibody or antigen binding portion thereof comprises two variable domains selected from the group disclosed above. Preferably, the CDR grafted antibody or antigen binding portion thereof comprises a human acceptor framework. More preferably the human acceptor framework is any one of the human acceptor frameworks disclosed above.

In a preferred embodiment, the binding protein is a humanized antibody or antigen binding portion thereof capable of binding an Aβ(1-42) globulomer. Preferably, the humanized antibody or antigen binding portion thereof comprises one or more CDRs disclosed above incorporated into a human antibody variable domain of a human acceptor framework. Preferably, the human antibody variable domain is a consensus human variable domain. More preferably, the human acceptor framework comprises at least one Framework Region amino acid substitution at a key residue, wherein the key residue is selected from the group consisting of a residue adjacent to a CDR; a glycosylation site residue; a rare residue; a residue capable of interacting with an Aβ(1-42) globulomer; a residue capable of interacting with a CDR; a canonical residue; a contact residue between heavy chain variable region and light chain variable region; a residue within a Vernier zone; and a residue in a region that overlaps between a Chothia-defined variable heavy chain CDR1 and a Kabat-defined first heavy chain framework. Preferably, the human acceptor framework human acceptor framework comprises at least one Framework Region amino acid substitution, wherein the amino acid sequence of the framework is at least 65% identical to the sequence of said human acceptor framework and comprises at least 52 amino acid residues identical to said human acceptor framework.

In a preferred embodiment, the binding protein is a humanized antibody or antigen binding portion thereof capable of binding to an Aβ(1-42) globulomer. Preferably the humanized antibody, or antigen binding portion, thereof comprises one or more CDRs disclosed above. More preferably the humanized antibody, or antigen binding portion thereof, comprises three or more CDRs disclosed above. Most preferably the humanized antibody, or antigen-binding portion thereof, comprises six CDRs disclosed above.

In another embodiment of the claimed invention, the humanized antibody or antigen binding portion thereof comprises at least one variable domain having an amino acid sequence selected from the group consisting of SEQ ID NO:1 and SEQ ID NO.:2. With respect to SEQ ID NO:1 (8F5 hum8 VL), based upon Kabat numbering, amino acid position 19 may be K; position 40 may be T; position 42 may be D; position 44 may be R; position 82A may be S; position 83 may be K; position 84 may be S; position 89 may be M, and J segment 107-109 “TLV” may be “STL”. In connection with SEQ ID NO:2 (8F5 hum7 VH), based upon Kabat numbering, amino acid position 7 may be T; position 14 may be S; position 15 may be L; position 17 may be D; position 18 may be Q; position 83 may be L; and position 87 may be F. More preferably, the humanized antibody or antigen-binding portion thereof comprises two variable domains selected from the group disclosed above. Most preferably, humanized antibody, or an antigen-binding portion thereof, comprises two variable domains, wherein said two variable domains have amino acid sequences selected from the group consisting of SEQ ID NOS.:11, 12 and 13 & SEQ ID NOS.:14, 15 and 16.

In a preferred embodiment, the binding protein disclosed above comprises a heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, and a human IgA constant domain. More preferably, the binding protein comprises SEQ ID NO:25, SEQ ID NO:26, SEQ ID NO:27 and SEQ ID NO:28.

The binding protein of the invention is capable of binding Aβ(1-42) globulomer. Preferably, the binding protein is a humanized antibody (e.g., 8F5) capable of modulating a biological function of an Aβ(1-42) globulomer. More preferably the binding protein is capable of neutralizing an Aβ(1-42) globulomer. Further, the humanized antibody binds with greater specificity to an amyloid beta (Aβ) protein globulomer than to an amyloid beta protein monomer. Thus, preferential binding is observed. The ratio of binding specificity to the globulomer versus the monomer is at least 1.4. In particular, the ratio is preferably at least about 1.4 to at least about 16.9. (A ratio of 1.0-17.5 including the endpoints) is also considered to fall within the scope of the present invention as well as decimal percentages thereof. For example, 1.1, 1.2, 1.3, . . . , 2.0, 2.1, 2.2 . . . , 17.1, 17.2, 17.3, 17.4, 17.5 as well as all full integers in between and percentages thereof are considered to fall within the scope of the present invention.) The amyloid beta protein monomer may be, for example, Aβ(1-42) monomer or Aβ(1-40) monomer.

In another embodiment, the binding protein of the invention has a dissociation constant (K_{D}) to an Aβ(1-42) globulomer of 1×10⁻⁶ M to 1×10⁻¹². Preferably, the antibody binds to an Aβ(1-42) globulomer with high affinity, for instance, with a K_{D} of 1×10⁻⁷ M or greater affinity (for example, with a K_{D} of 3×10⁻⁸ M or greater affinity), with a K_{D} of 1×10⁻⁹ M or greater affinity (for example, 3×10⁻¹⁰ M or greater affinity), with a K_{D} or 1×10⁻¹⁰ M or greater affinity (for example, with a K_{D} of 3×10⁻¹¹ M or greater affinity) or with a K_{D} of 1×10⁻¹¹ M or greater affinity.

One embodiment of the invention provides an antibody construct comprising any one of the binding proteins disclosed above and a linker polypeptide or an immunoglobulin. In a preferred embodiment, the antibody construct is selected from the group consisting of an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, a dual variable domain antibody and a bispecific antibody. In a preferred embodiment, the antibody construct comprises a heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, and a human IgA constant domain. More preferably, the antibody construct comprises (SEQ ID NO:25 and SEQ ID NO:26) or (SEQ ID NO:27 and SEQ ID NO:28). In another embodiment the invention provides an antibody conjugate comprising an the antibody construct disclosed above and an agent an agent selected from the group consisting of; an immunoadhension molecule, an imaging agent, a therapeutic agent, and a cytotoxic agent. In a preferred embodiment the imaging agent selected from the group consisting of a radiolabel, an enzyme, a fluorescent label, a luminescent label, a bioluminescent label, a magnetic label, and biotin. More preferably the imaging agent is a radiolabel selected from the group consisting of: ³H, ¹⁴C, ³⁵S, ⁹⁰Y, ⁹⁹Tc, 111In, 125I, ¹³¹I, ¹⁷⁷Lu, ¹⁶⁶Ho, and ¹⁵³Sm. In a preferred embodiment the therapeutic or cytotoxic agent is selected from the group consisting of; an anti-metabolite, an alkylating agent, an antibiotic, a growth factor, a cytokine, an anti-angiogenic agent, an anti-mitotic agent, an anthracycline, toxin, and an apoptotic agent.

In another embodiment, the antibody construct is glycosylated. Preferably, the glycosylation is a human glycosylation pattern.

In a further embodiment, the binding protein, antibody construct or antibody conjugate disclosed above exists as a crystal. Preferably, the crystal is a carrier-free pharmaceutical controlled release crystal. In a preferred embodiment, the crystallized binding protein, crystallized antibody construct or crystallized antibody conjugate has a greater half life in vivo than its soluble counterpart. In another preferred embodiment, the crystallized binding protein, crystallized antibody construct or crystallized antibody conjugate retains biological activity after crystallization.

One aspect of the invention pertains to an isolated nucleic acid encoding the binding protein, antibody construct or antibody conjugate disclosed above. A further embodiment provides a vector comprising the isolated nucleic acid disclosed above wherein said vector is selected from the group consisting of pcDNA; pTT (Durocher et al., Nucleic Acids Research 2002, Vol 30, No. 2); pTT3 (pTT with additional multiple cloning site; pEFBOS (Mizushima, S. and Nagata, S., (1990) Nucleic Acids Research, Vol. 18, No. 17); pBV; pJV; and pBJ.

The present invention also encompasses a host cell which is transformed with the vector disclosed above. Preferably, the host cell is a prokaryotic cell. More preferably, the host cell is E. coli. In a related embodiment, the host cell is an eukaryotic cell. Preferably, the eukaryotic cell is selected from the group consisting of protist cell, animal cell, plant cell and fungal cell. More preferably, the host cell is a mammalian cell including, but not limited to, CHO and COS; or a fungal cell such as Saccharomyces cerevisiae; or an insect cell such as Sf9.

Additionally, the present invention includes a method of producing a binding protein that binds Aβ(1-42) globulomer, comprising culturing any one of the host cells disclosed above in a culture medium under conditions and for a time sufficient to produce a binding protein that binds Aβ(1-42). Another embodiment provides a binding protein produced according to the method disclosed above.

One embodiment provides a composition for the release of a binding protein, as defined herein, wherein the composition comprises a formulation which in turn comprises a crystallized binding protein, crystallized antibody construct or crystallized antibody conjugate as disclosed above and an ingredient; and at least one polymeric carrier. Preferably the polymeric carrier is a polymer selected from one or more of the group consisting of: poly (acrylic acid), poly (cyanoacrylates), poly (amino acids), poly (anhydrides), poly (depsipeptide), poly (esters), poly (lactic acid), poly (lactic-co-glycolic acid) or PLGA, poly (b-hydroxybutryate), poly (caprolactone), poly (dioxanone); poly (ethylene glycol), poly ((hydroxypropyl) methacrylamide, poly [(organo)phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polyeaccharides, blends and copolymers thereof. Preferably the ingredient is selected from the group consisting of albumin, sucrose, trehalose, lactitol, gelatin, hydroxypropyl-cyclodextrin, methoxypolyethylene glycol and polyethylene glycol. Another embodiment provides a method for treating a mammal comprising the step of administering to the mammal an effective amount of the composition disclosed above.

The invention also encompasses a pharmaceutical composition comprising a binding protein, antibody construct or antibody conjugate as disclosed above and a pharmaceutically acceptable carrier. In a further embodiment, the pharmaceutical composition comprises at least one additional therapeutic agent for treating a disorder in which activity is detrimental. Preferably the additional therapeutic agent is selected from the group consisting of a cholesterinase inhibitor, a partial NMDA receptor blocker, a glycosaminoglycan mimetic, an inhibitor or allosteric modulator of gamma secretase, a luteinizing hormone blockade gonadotropin releasing hormone agonist, a serotinin 5-HT1A receptor antagonist, a chelating agent, a neuronal selective L-type calcium channel blocker, an immunomodulator, an amyloid fibrillogenesis inhibitor or amyloid protein deposition inhibitor, a PDE4 inhibitor, a histamine agonist, a receptor protein for advanced glycation end products, a PARP stimulator, a serotonin 6-receptor antagonist, a 5-HT4 receptor agonist, a human steroid, a glucose uptake stimulant which enhances neuronal metabolism, a selective CB1 antagonist, a partial agonist at benzodiazepine receptors, an amyloid beta production antagonist or inhibitor, an amyloid beta deposition inhibitor, a NNR alpha-7 partial antagonist, a therapeutic targeting PDE4, a RNA translation inhibitor, a muscarinic agonist, a nerve growth factor receptor agonist, a NGF receptor agonist and a gene therapy modulator.

In another aspect, the invention provides a method for inhibiting activity of Aβ(1-42) globulomer comprising contacting Aβ(1-42) globulomer with a binding protein disclosed above such that Aβ(1-42) globulomer activity is inhibited. In a related aspect, the invention provides a method for inhibiting human Aβ(1-42) globulomer activity in a human subject suffering from a disorder in which Aβ(1-42) globulomer activity is detrimental, comprising administering to the human subject a binding protein disclosed above such that Aβ(1-42) globulomer activity in the human subject is inhibited and treatment is achieved. Examples of conditions or diseases which may be treated using this method include but are not limited to Alpha1-antitrypsin-deficiency, C1-inhibitor deficiency angioedema, Antithrombin deficiency thromboembolic disease, Kuru, Creutzfeld-Jacob disease/scrapie, Bovine spongifor encephalopathy, Gerstmann-Straussler-Scheinker disease, Fatal familial insomnia, Huntington's disease, Spinocerebellar ataxia, Machado-Joseph atrophy, Dentato-rubro-pallidoluysian atrophy, Frontotemporal dementia, Sickle cell anemia, Unstable hemoglobin inclusion-body hemolysis, Drug-induced inclusion body hemolysis, Parkinson's disease, Systemic AL amyloidosis, Nodular AL amyloidosis, Systemic AA amyloidosis, Prostatic amyloid, Hemodialysis amyloidosis, Hereditary (Icelandic) cerebral angiopathy, Huntington's disease, Familial visceral amyloid, Familial visceral polyneuropathy, Familial visceral amyloidosis, Senile systemic amyloidosis, Familial amyloid neurophathy, Familial cardiac amyloid, Alzheimer's disease, Down's syndrome, Medullary carcinoma thyroid and Type 2 diabetes mellitus (T2DM). Preferably the disorder is selected from an amyloidosis such as, for example, Alzheimer's Disease or Down's Syndrome.

In another aspect the invention provides a method of treating a patient suffering from a disorder in which Aβ(1-42) globulomer is detrimental comprising the step of administering any one of the binding proteins disclosed above before, concurrent, or after the administration of a second agent, as discussed above. In a preferred embodiment, the second agent is selected from the group consisting of a small molecule or a biologic (i.e., see list of additional therapeutic agents presented above (e.g., an additional antibody, a cholinesterase inhibitor, a partial NMDA receptor blocker, etc.)).

In a preferred embodiment, the pharmaceutical compositions disclosed above are administered to the subject by at least one mode selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

One aspect of the invention provides at least one Aβ(1-42) globulomer anti-idiotype antibody to at least one Aβ(1-42) globulomer binding protein of the present invention. The anti-idiotype antibody includes any protein or peptide containing molecule that comprises at least a portion of an immunoglobulin molecule such as, but not limited to, at least one complementarily determining region (CDR) of a heavy or light chain or a ligand binding portion thereof, a heavy chain or light chain variable region, a heavy chain or light chain constant region, a framework region, or; any portion thereof, that can be incorporated into a binding protein of the present invention.

### BRIEF DESCRIPTION OF THE FIGURES

FIG. 1(A) illustrates the nucleotide sequence (SEQ ID NO:3) of the variable heavy chain of humanized antibody 8F5, and FIG. 1(B) illustrates the amino acid sequence of the variable heavy chain (SEQ ID NO:1) encoded by this nucleotide sequence. FIG. 1(C) illustrates the nucleotide sequence (SEQ ID NO:4) of the variable light chain of humanized antibody 8F5, and FIG. 1(D) illustrates the amino acid sequence (SEQ ID NO:2) of the variable light chain encoded by this nucleotide sequence. (All CDR regions are underlined.)

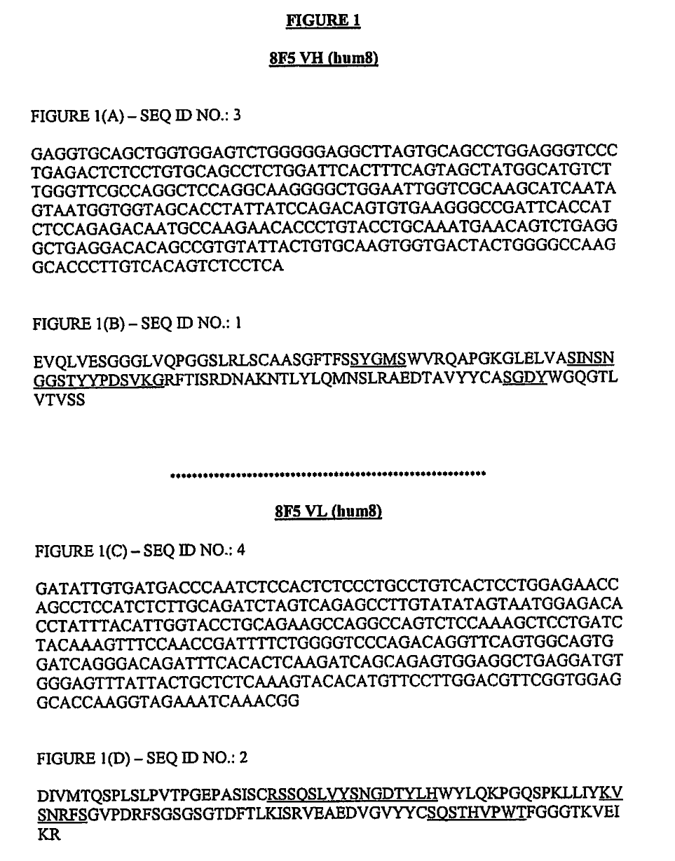

FIG. 2(A) shows an SDS PAGE of standard proteins (molecular marker proteins, lane 1); Aβ(1-42) fibril preparation; control (lane 2); Aβ(1-42) fibril preparation+mAb 8F5 hum8, 20 h, 37° C., supernatant (lane 3); Aβ(1-42) fibril preparation+mAb 8F5 hum8, 20 h, 37° C., pellet (lane 4); Aβ(1-42) fibril preparation+mAb 6E10, 20 h, 37° C., supernatant (lane 5); Aβ(1-42) fibril preparation+mAb 6E10, 20 h 37° C., pellet (lane 6); Aβ(1-42) fibril preparation+mAb IgG2a, 20 h 37° C., supernatant (lane 7); Aβ(1-42) fibril preparation+mAb IgG2a, 20 h 37° C., pellet (lane 8), and FIG. 2(B) shows the results of the quantitative analysis of mAbs bound to Aβ-fibrils in percent of total antibody.

FIG. 3 illustrates the binding of the biotinylated mouse 8F5 antibody to the Aβ(1-42) globulomer. In particular, the binding of the biotinylated mouse 8F5 antibody is inhibited by increasing amounts of unlabeled mouse antibody (HYB) or humanized antibody (HUM8).

FIG. 4 illustrates the alignment of the 8F5VH region amino acid sequences. The amino acid sequences of 8F5VH (SEQ ID NO:100), Hu8F5VHv1 (SEQ ID NO:101), Hu8F5VHv2 (SEQ ID NO:102), and the human YSE′CL (SEQ ID NO:103) and JH4 segments are shown in single letter code. The CDR sequences based on the definition of Kabat, E. A., et al. (1991) are underlined in the mouse 8F5VH sequence. The CDR sequences in the acceptor human VH segment are omitted in the figure. The single underlined amino acids in the Hu8F5VHv1 and Hu8F5VHv2 sequences are predicted to contact the CDR sequences, and therefore have been substituted with the corresponding mouse residues. The double underlined amino acids in the Hu8F5VHv1 and Hu8F5VHv2 sequences have been changed to the consensus amino acids in the same human VH subgroup to eliminate potential immunogenicity.

FIG. 5 illustrates the alignment of the 8F5VL region amino acid sequences. The amino acid sequences of 8F5VL (SEQ ID NO:104), Hu8F5VL (SEQ ID NO:105), and the human TR1.37′CL (SEQ ID NO:106) and JK4 segments are shown in single letter code. The CDR sequences based on the definition of Kabat, E. A., et al. (1991) are underlined in the mouse 8F5VL sequence. The CDR sequences in the acceptor human VL segment are omitted in the figure. The single underlined amino acid in the Hu8F5VL sequence is predicted to contact the CDR sequences, and therefore has been substituted with the corresponding mouse residue. The double underlined amino acids in the Hu8F5VL sequence have been changed to the consensus amino acids in the same human VL subgroup to eliminate potential immunogenicity.

FIG. 6 illustrates the nucleotide sequence (SEQ ID NO:107) and deduced amino acid sequence (SEQ ID NO:108) of the heavy chain variable region of Hu8F5VHv1 in the mini exon. The signal peptide sequence is in italics. The mature heavy chain begins with a glutamate residue (indicated in bold). The CDRs based on the definition of Kabat, E. A., et al. (1991) are underlined. The sequence is flanked by unique MluI (ACGCGT) and XbaI (TCTAGA) sites.

FIG. 7 illustrates the nucleotide sequence (SEQ ID NO:109) and deduced amino acid sequence (SEQ ID NO:110) of the heavy chain variable region of Hu8F5VHv2 in the mini exon. The signal peptide sequence is in italics. The mature heavy chain begins with a glutamate residue (indicated in bold). The CDRs based on the definition of Kabat, E. A., et al. (1991) are underlined. The sequence is flanked by unique MluI (ACGCGT) and XbaI (TCTAGA) sites.

FIG. 8 illustrates the nucleotide sequence (SEQ ID NO:111) and deduced amino acid sequence (SEQ ID NO:112) of the light chain variable region of Hu8F5VL in the mini exon. The signal peptide sequence is in italics. The mature light chain begins with an aspartate residue (indicated in bold). The CDRs based on the definition of Kabat, E. A., et al. (1991) are underlined. The sequence is flanked by unique MluI (ACGCGT) and XbaI (TCTAGA) sites.

FIG. 9 illustrates the restriction maps of expression plasmids pHu8F5VHv1-Cg1 and pVk-Hu8F5VL.

FIG. 10 illustrates the competition ELISA to compare the relative binding affinities of various 8F5 antibodies to human A-beta oligomer antigen (1-42). The binding of biotinylated Mu8F5 to immobilized human A-beta oligomer antigen (1-42) was analyzed in the presence of different amounts of Mu8F5 and Hu8F5 competitor antibodies as described in Example I.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

In order that the present invention may be more readily understood, select terms are defined below.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “recovering” as used herein, refers to the process of rendering a chemical species such as a polypeptide substantially free of naturally associated components by isolation, e.g., using protein purification techniques well known in the art.

The term “Aβ(X-Y)” herein refers to the amino acid sequence from amino acid position “X” to amino acid position “Y” of the human amyloid β protein including both X and Y and, in particular, refers to the amino acid sequence from amino acid position 1 to amino acid position 42 of the amino acid sequence DAEFRHDSGY EVHHQKLVFF AEDVGSNKGA IIGLMVGGVV IA (SEQ ID NO:29) or any of its naturally occurring variants, in particular, those with at least one mutation selected from the group consisting of A2T, H6R, D7N, A21G (“Flemish”), E22G (“Arctic”), E22Q (“Dutch”), E22K (“Italian”), D23N (“Iowa”), A42T and A42V wherein the numbers are relative to the start position of the Aβ peptide, including both position X and position Y or a sequence with up to three additional amino acid substitutions none of which may prevent globulomer formation. An “additional” amino acid substitution is defined herein as any deviation from the canonical sequence that is not found in nature.

More specifically, the term “Aβ(1-42)” herein refers to the amino acid sequence from amino acid position 1 to amino acid position 42 of the human amyloid β protein including both 1 and 42 and, in particular, refers to the amino acid sequence from amino acid position 1 to amino acid position 42 of the amino acid sequence DAEFRHDSGY EVHHQKLVFF AEDVGSNKGA IIGLMVGGVV IA (i.e., the full sequence corresponding to amino acid positions 1 to 42; SEQ ID NO:29) or any of its naturally occurring variants. Such variants may be, for example, those with at least one mutation selected from the group consisting of A2T, H6R, D7N, A21G (“Flemish”), E22G (“Arctic”), E22Q (“Dutch”), E22K (“Italian”), D23N (“Iowa”), A42T and A42V wherein the numbers are relative to the start of the Aβ peptide, including both 1 and 42 or a sequence with up to three additional amino acid substitutions none of which may prevent globulomer formation. Likewise, the term “Aβ(1-40)” herein refers to the amino acid sequence from amino acid position 1 to amino acid position 40 of the human amyloid β protein including both 1 and 40 and refers, in particular, to the amino acid sequence from amino acid position 1 to amino acid position 40 of the amino acid sequence DAEFRHDSGY EVHHQKLVFF AEDVGSNKGA IIGLMVGGVV (SEQ ID NO:30) or any of its naturally occurring variants. Such variants include, for example, those with at least one mutation selected from the group consisting of A2T, H6R, D7N, A21G (“Flemish”), E22G (“Arctic”), E22Q (“Dutch”), E22K (“Italian”), and D23N (“Iowa”) wherein the numbers are relative to the start position of the Aβ peptide, including both 1 and 40 or a sequence with up to three additional amino acid substitutions none of which may prevent globulomer formation.

The term “Aβ(X-Y) globulomer” (also known as “Aβ(X-Y) globular oligomer”) herein refers to a soluble, globular, non-covalent association of Aβ(X-Y) peptides, as defined above, possessing homogeneity and distinct physical characteristics. The Aβ(X-Y) globulomers are stable, non-fibrillar, oligomeric assemblies of Aβ(X-Y) peptides which are obtainable by incubation with anionic detergents. In contrast to monomer and fibrils, these globulomers are characterized by defined assembly numbers of subunits (e.g., early assembly forms, n=3-6, oligomers A″, and late assembly forms, n=12-14, “ oligomers B”, as described in International Application Publication No. WO 04/067561 herein incorporated by reference). The globulomers have a 3-dimensional globular type structure (“molten globule”, see Barghorn et al., 2005, J Neurochem, 95, 834-847). They may be further characterized by one or more of the following features:

cleavability of N-terminal amino acids X-23 with promiscuous proteases (such as thermolysin or endoproteinase GluC) yielding truncated forms Aβ(X-Y) globulomers;

non-accessibility of C-terminal amino acids 24-Y with promiscuous proteases and antibodies; and

truncated forms of these Aβ(X-Y) globulomers maintain the 3-dimensional core structure of the globulomers with a better accessibility of the core epitope Aβ(20-Y) in its globulomer conformation.

According to the invention and, in particular, for the purpose of assessing the binding affinities of the antibodies of the present invention, the term “Aβ(X-Y) globulomer” herein refers to a product which is obtainable by a process as described in International Application Publication No. WO 04/067561, which is incorporated herein in its entirety by reference. The process comprises unfolding a natural, recombinant or synthetic Aβ(X-Y) peptide or a derivative thereof; exposing the at least partially unfolded Aβ(X-Y) peptide or derivative thereof to a detergent, reducing the detergent action and continuing incubation.

For the purpose of unfolding the peptide, hydrogen bond-breaking agents such as, for example, hexafluoroisopropanol (HFIP) may be allowed to act on the protein. Times of action of a few minutes, for example about 10 to 60 minutes, are sufficient when the temperature of action is from about 20 to 50° C. and, in particular, about 35 to 40° C. Subsequent dissolution of the residue evaporated to dryness, preferably in concentrated form, in suitable organic solvents miscible with aqueous buffers such as, for example, dimethyl sulfoxide (DMSO), results in a suspension of the at least partially unfolded peptide or derivative thereof which can be used subsequently. If required, the stock suspension may be stored at low temperature, for example, at about −20° C. for an interim period.

Alternatively, the peptide or the derivative thereof may be taken up in slightly acidic, preferably aqueous, solution, for example, a solution of about 10 mM aqueous HCl. After an incubation time of approximately a few minutes, insoluble components are removed by centrifugation. A few minutes at 10,000 g is expedient. These method steps are preferably carried out at room temperature, i.e., a temperature in the range of from 20 to 30° C. The supernatant obtained after centrifugation contains the Aβ(X-Y) peptide or a derivative thereof and may be stored at low temperature, for example at about −20° C., for an interim period.

The following exposure to a detergent relates to oligomerization of the peptide or the derivative thereof to give the intermediate type of oligomers (in International Application Publication No. WO 04/067561 referred to as oligomers A). For this purpose, a detergent is allowed to act on the, optionally, at least partially unfolded peptide or derivative thereof until sufficient intermediate oligomer has been produced. Preference is given to using ionic detergents, in particular, anionic detergents.

According to a particular embodiment, a detergent of the formula (I):

R-X,

is used, in which the radical “R” is unbranched or branched alkyl having from 6 to 20 and preferably 10 to 14 carbon atoms or unbranched or branched alkenyl having from 6 to 20 and preferably 10 to 14 carbon atoms, and the radical “X” is an acidic group or salt thereof with X being preferably selected from among —COO⁻M⁺, —SO₃⁻M⁺ and is, most preferably, —OSO₃⁻M⁺ and M⁺ is a hydrogen cation or an inorganic or organic cation preferably selected from alkali metal cations, alkaline earth metal cations and ammonium cations.

Most advantageous are detergents of the formula (I) in which R is an unbranched alkyl of which alk-1-yl radicals must be mentioned, in particular. Particular preference is given to sodium dodecyl sulfate (SDS). Lauric acid and oleic acid can also be used advantageously. The sodium salt of the detergent lauroylsarcosin (also known as sarkosyl NL-30 or Gardol®) is also particularly advantageous.

The time of detergent action, in particular, depends on whether, and if yes, to what extent the peptide or derivative thereof subjected to oligomerization has unfolded. If, according to the unfolding step, the peptide or derivative thereof has been treated beforehand with a hydrogen bond-breaking agent (i.e., in particular with hexafluoroisopropanol), times of action in the range of a few hours, advantageously from about 1 to 20 and, in particular, from about 2 to 10 hours, are sufficient when the temperature of action is about 20 to 50° C. and, in particular, from about 35 to 40° C. If a less unfolded or an essentially not unfolded peptide or derivative thereof is the starting point, correspondingly longer times of action are expedient. If the peptide or derivative thereof has been pretreated, for example, according to the procedure indicated above as an alternative to the HFIP treatment or said peptide or derivative thereof is directly subjected to oligomerization, times of action in the range from about 5 to 30 hours and, in particular, from about 10 to 20 hours are sufficient when the temperature of action is from about 20 to 50° C. and, in particular, from about 35 to 40° C. After incubation, insoluble components are advantageously removed by centrifugation. A few minutes at 10,000 g is expedient.

The detergent concentration to be chosen depends on the detergent used. If SDS is used, a concentration in the range from 0.01 to 1% by weight, preferably, from 0.05 to 0.5% by weight, for example, of about 0.2% by weight, proves expedient. If lauric acid or oleic acid is used, somewhat higher concentrations are expedient, for example, in a range from 0.05 to 2% by weight, preferably, from 0.1 to 0.5% by weight, for example, of about 0.5% by weight.

The detergent action should take place at a salt concentration approximately in the physiological range. Thus, in particular NaCl concentrations in the range from 50 to 500 mM, preferably, from 100 to 200 mM and, more particularly, at about 140 mM are expedient.

The subsequent reduction of the detergent action and continuation of incubation relates to further oligomerization to give the Aβ(X-Y) globulomer of the invention (in International Application Publication No. WO 04/067561 referred to as oligomer B). Since the composition obtained from the preceding step regularly contains detergent and a salt concentration in the physiological range, it is then expedient to reduce detergent action and, preferably, also salt concentration. This may be carried out by reducing the concentration of detergent and salt, for example, by diluting expediently with water or a buffer of lower salt concentration, for example, Tris-HCl, pH 7.3. Dilution factors in the range from about 2 to 10, advantageously, in the range from about 3 to 8 and, in particular, of about 4, have proved suitable. The reduction in detergent action may also be achieved by adding substances which can neutralize this detergent action. Examples of these include substances capable of complexing the detergents, like substances capable of stabilizing cells in the course of purification and extraction measures, for example, particular EO/PO block copolymers, in particular, the block copolymer under the trade name Pluronic® F 68. Alkoxylated and, in particular, ethoxylated alkyl phenols such as the ethoxylated t-octylphenols of the Triton® X series, in particular, Triton® X100, 3-(3-cholamidopropyldimethylammonio)-1-propanesulfonate (CHAPS®) or alkoxylated and, in particular, ethoxylated sorbitan fatty esters such as those of the Tween® series, in particular, Tween® 20, in concentration ranges around or above the particular critical micelle concentration, may be equally used.

Subsequently, the solution is incubated until sufficient Aβ(X-Y) globulomer has been produced. Times of action in the range of several hours, preferably, in the range from about 10 to 30 hours and, in particular, in the range from about 15 to 25 hours, are sufficient when the temperature of action is about 20 to 50° C. and, in particular, about 35 to 40° C. The solution may then be concentrated and possible residues may be removed by centrifugation. Again, a few minutes at 10,000 g proves expedient. The supernatant obtained after centrifugation contains an Aβ(X-Y) globulomer as described herein.

An Aβ(X-Y) globulomer can be finally recovered, e.g., by ultrafiltration, dialysis, precipitation or centrifugation. It is further preferred if electrophoretic separation of the Aβ(X-Y) globulomers under denaturing conditions, e.g. by SDS-PAGE, produces a double band (e.g., with an apparent molecular weight of 38/48 kDa for Aβ(1-42)) and especially preferred if upon glutardialdehyde treatment of the oligomers, before separation, these two bands are merged into one. It is also preferred if size exclusion chromatography of the globulomers results in a single peak (e.g., corresponding to a molecular weight of approximately 60 kDa for Aβ(1-42)). Starting from Aβ(1-42) peptide, the process is, in particular, suitable for obtaining Aβ(1-42) globulomers. Preferably, the globulomer shows affinity to neuronal cells and also exhibits neuromodulating effects. A “neuromodulating effect” is defined as a long-lasting inhibitory effect of a neuron leading to a dysfunction of the neuron with respect to neuronal plasticity.

According to another aspect of the invention, the term “Aβ(X-Y) globulomer” herein refers to a globulomer consisting essentially of Aβ(X-Y) subunits, wherein it is preferred if, on average, at least 11 of 12 subunits are of the Aβ(X-Y) type, more preferred, if less than 10% of the globulomers comprise any non-Aβ(X-Y) peptides and, most preferred, if the content of non-Aβ(X-Y) peptides in the preparation is below the detection threshold. More specifically, the term “Aβ(1-42) globulomer” herein refers to a globulomer comprising Aβ(1-42) units as defined above; the term “Aβ(12-42) globulomer” herein refers to a globulomer comprising Aβ(12-42) units as defined above; and the term “Aβ(20-42) globulomer” herein refers to a globulomer comprising Aβ(20-42) units as defined above.

The term “cross-linked Aβ(X-Y) globulomer” herein refers to a molecule obtainable from an Aβ(X-Y) globulomer as described above by cross-linking, preferably, chemically cross-linking, more preferably, aldehyde cross-linking and, most preferably, glutardialdehyde cross-linking of the constituent units of the globulomer. In another aspect of the invention, a cross-linked globulomer is essentially a globulomer in which the units are at least partially joined by covalent bonds, rather than being held together by non-covalent interactions only.

The term “Aβ(X-Y) globulomer derivative” herein refers, in particular, to a globulomer that is labelled by being covalently linked to a group that facilitates detection, preferably, a fluorophore, e.g., fluorescein isothiocyanate, phycoerythrin, Aequorea victoria fluorescent protein, Dictyosoma fluorescent protein or any combination or fluorescence-active derivatives thereof; a chromophore; a chemoluminophore, e.g., luciferase, preferably Photinus pyralis luciferase, Vibrio fischeri luciferase, or any combination or chemoluminescence-active derivatives thereof; an enzymatically active group, e.g., peroxidase such as horseradish peroxidase, or an enzymatically active derivative thereof; an electron-dense group, e.g., a heavy metal containing group such as a gold containing group; a hapten, e.g., a phenol derived hapten; a strongly antigenic structure, e.g., peptide sequence predicted to be antigenic such as by the algorithm of Kolaskar and Tongaonkar; an aptamer for another molecule; a chelating group, e.g., hexahistidinyl; a natural or nature-derived protein structure mediating further specific protein-protein interactions, e.g., a member of the fos/jun pair; a magnetic group, e.g., a ferromagnetic group; or a radioactive group such as a group comprising ¹H, ¹⁴C, ³P, ³S or ¹²⁵I or any combination thereof; or to a globulomer flagged by being covalently or by non-covalently linked by high-affinity interaction, preferably, covalently linked to a group that facilitates inactivation, sequestration, degradation and/or precipitation, preferably, flagged with a group that promotes in vivo degradation, more preferably, with ubiquitin, where it is particularly preferred if this flagged oligomer is assembled in vivo; or to a globulomer modified by any combination of the above. Such labelling and flagging groups and methods for attaching them to proteins are known in the art. Labelling and/or flagging may be performed before, during or after globulomerization. In another aspect of the invention, a globulomer derivative is a molecule obtainable from a globulomer by a labelling and/or flagging reaction. Correspondingly, the term “Aβ(X-Y) monomer derivative” herein refers, in particular, to an Aβ monomer that is labelled or flagged as described for the globulomer.

The term “greater affinity” herein refers to a degree of interaction where the equilibrium between unbound antibody and unbound globulomer, on the one hand, and antibody-globulomer complex, on the other, is further in favor of the antibody-globulomer complex. Likewise, the term “smaller affinity” herein refers to a degree of interaction where the equilibrium between unbound antibody and unbound globulomer, on the one hand, and antibody-globulomer complex, on the other, is further in favor of the unbound antibody and unbound globulomer.

The term “Aβ(X-Y) monomer” herein refers to the isolated form of the Aβ(X-Y) peptide, preferably, a form of the Aβ(X-Y) peptide which is not engaged in essentially non-covalent interactions with other Aβ peptides. Practically, the Aβ(X-Y) monomer is usually provided in the form of an aqueous solution. Preferably, the aqueous monomer solution contains 0.05% to 0.2%, more preferably, about 0.1% NaOH when used, for instance, for determining the binding affinity of the antibody of the present invention. In another preferable situation, the aqueous monomer solution contains 0.05% to 0.2%, more preferably, about 0.1% NaOH. When used, it may be expedient to dilute the solution in an appropriate manner. Further, it is usually expedient to use the solution within 2 hours, in particular, within 1 hour, and, especially, within 30 minutes after its preparation.

The term “fibril” herein refers to a molecular structure that comprises assemblies of non-covalently associated, individual Aβ(X-Y) peptides which show fibrillary structure under the electron microscope, which bind Congo red, exhibit birefringence under polarized light and whose X-ray diffraction pattern is a cross-β structure. The fibril may also be defined as a molecular structure obtainable by a process that comprises the self-induced polymeric aggregation of a suitable Aβ peptide in the absence of detergents, e.g., in 0.1 M HCl, leading to the formation of aggregates of more than 24, preferably, more than 100 units. This process is well known in the art. Expediently, Aβ(X-Y) fibril is used in the form of an aqueous solution. In a particularly preferred embodiment of the invention, the aqueous fibril solution is made by dissolving the Aβ peptide in 0.1% NH₄OH, diluting it 1:4 with 20 mM NaH₂PO₄, 140 mM NaCl, pH 7.4, followed by readjusting the pH to 7.4, incubating the solution at 37° C. for 20 h, followed by centrifugation at 10000 g for 10 min and resuspension in 20 mM NaH₂PO₄, 140 mM NaCl, pH 7.4.

The term “Aβ(X-Y) fibril” herein refers to a fibril comprising Aβ(X-Y) subunits where it is preferred if, on average, at least 90% of the subunits are of the Aβ(X-Y) type, more preferred, if at least 98% of the subunits are of the Aβ(X-Y) type and, most preferred, if the content of non-Aβ(X-Y) peptides is below the detection threshold.

Turning back to antibody 8F5, this Aβ(1-42) globulomer-specific antibody recognizes predominantly Aβ(1-42) globulomer forms and not standard preparations of Aβ(1-40) monomers, Aβ(1-42) monomers, Aβ-fibrils or sAPP (i.e, Aβprecursor) in contrast to, for example, competitor antibodies such as m266 and 3D6. Such specificity for globulomers is important because specifically targeting the globulomer form of Aβ with a globulomer preferential antibody such as, for example, 8F5, will: 1) avoid targeting insoluble amyloid deposits, binding to which may account for inflammatory side effects observed during immunizations with insoluble Aβ; 2) spare Aβ monomer and APP that are reported to have precognitive physiological functions (Plan et al., J. of Neuroscience 23:5531-5535 (2003); and 3) increase the bioavailability of the antibody, as it would not be shaded or inaccessible through extensive binding to insoluble deposits.

The subject invention also includes isolated nucleotide sequences (or fragments thereof) encoding the variable light and heavy chains of antibody 8F5 as well as those nucleotide sequences (or fragments thereof) having sequences comprising, corresponding to, identical to, hybridizable to, or complementary to, at least about 70% (e.g., 70% 71%, 72%, 73%, 74%, 75%, 76%, 77%, 78% or 79%), preferably at least about 80% (e.g., 80%, 81%, 82%, 83%, 84%, 85%, 86%, 87%, 88% or 89%), and more preferably at least about 90% (e.g, 91%, 92%, 93%, 94%, 95%, 96%, 97%, 98%, 99% or 100%) identity to these encoding nucleotide sequences. (All integers (and portions thereof) between and including 70% and 100% are considered to be within the scope of the present invention with respect to percent identity.) Such sequences may be derived from any source (e.g., either isolated from a natural source, produced via a semi-synthetic route, or synthesized de novo). In particular, such sequences may be isolated or derived from sources other than described in the examples (e.g., bacteria, fungus, algae, mouse or human).

In addition to the nucleotide sequences described above, the present invention also includes amino acid sequences of the variable light and heavy chains of antibody 8F5 (or fragments of these amino acid sequences). Further, the present invention also includes amino acid sequences (or fragments thereof) comprising, corresponding to, identical to, or complementary to at least about 70% (e.g., 70%, 71%, 72%, 73%, 74%, 75%, 76%, 77%, 78% or 79%), preferably at least about 80% (e.g., 80% 81%, 82%, 83%, 84%, 85%, 86%, 87%, 88% or 89%), and more preferably at least about 90% identity (e.g., 90%, 91%, 92%, 93%, 94%, 95%, 96%, 97%, 98%, 99% or 100%), to the amino acid sequences of the proteins of the present invention. (Again, all integers (and portions thereof) between and including 70% and 100% (as recited in connection with the nucleotide sequence identities noted above) are also considered to be within the scope of the present invention with respect to percent identity.)

For purposes of the present invention, a “fragment” of a nucleotide sequence is defined as a contiguous sequence of approximately at least 6, preferably at least about 8, more preferably at least about 10 nucleotides, and even more preferably at least about 15 nucleotides corresponding to a region of the specified nucleotide sequence.

The term “identity” refers to the relatedness of two sequences on a nucleotide-by-nucleotide basis over a particular comparison window or segment. Thus, identity is defined as the degree of sameness, correspondence or equivalence between the same strands (either sense or antisense) of two DNA segments (or two amino acid sequences). “Percentage of sequence identity” is calculated by comparing two optimally aligned sequences over a particular region, determining the number of positions at which the identical base or amino acid occurs in both sequences in order to yield the number of matched positions, dividing the number of such positions by the total number of positions in the segment being compared and multiplying the result by 100. Optimal alignment of sequences may be conducted by the algorithm of Smith & Waterman, Appl. Math. 2:482 (1981), by the algorithm of Needleman & Wunsch, J. Mol. Biol. 48:443 (1970), by the method of Pearson & Lipman, Proc. Natl. Acad. Sci. (USA) 85:2444 (1988) and by computer programs which implement the relevant algorithms (e.g., Clustal Macaw Pileup

(http://cmgm.stanford.edu/biochem218/11Multiple.pdf; Higgins et al., CABIOS. 5L151-153 (1989)), FASTDB (Intelligenetics), BLAST (National Center for Biomedical Information; Altschul et al., Nucleic Acids Research 25:3389-3402 (1997)), PILEUP (Genetics Computer Group, Madison, Wis.) or GAP, BESTFIT, FASTA and TFASTA (Wisconsin Genetics Software Package Release 7.0, Genetics Computer Group, Madison, Wis.). (See U.S. Pat. No. 5,912,120.)

For purposes of the present invention, “complementarity” is defined as the degree of relatedness between two DNA segments. It is determined by measuring the ability of the sense strand of one DNA segment to hybridize with the anti-sense strand of the other DNA segment, under appropriate conditions, to form a double helix. A “complement” is defined as a sequence which pairs to a given sequence based upon the canonic base-pairing rules. For example, a sequence A-G-T in one nucleotide strand is “complementary” to T-C-A in the other strand.

In the double helix, adenine appears in one strand, thymine appears in the other strand. Similarly, wherever guanine is found in one strand, cytosine is found in the other. The greater the relatedness between the nucleotide sequences of two DNA segments, the greater the ability to form hybrid duplexes between the strands of the two DNA segments.

“Similarity” between two amino acid sequences is defined as the presence of a series of identical as well as conserved amino acid residues in both sequences. The higher the degree of similarity between two amino acid sequences, the higher the correspondence, sameness or equivalence of the two sequences. (“Identity between two amino acid sequences is defined as the presence of a series of exactly alike or invariant amino acid residues in both sequences.) The definitions of “complementarity”, “identity” and “similarity” are well known to those of ordinary skill in the art.

“Encoded by” refers to a nucleic acid sequence which codes for a polypeptide sequence, wherein the polypeptide sequence or a portion thereof contains an amino acid sequence of at least 3 amino acids, more preferably at least 8 amino acids, and even more preferably at least 15 amino acids from a polypeptide encoded by the nucleic acid sequence.

“Biological activity” as used herein, refers to all inherent biological properties of the Aβ(1-42) region of the globulomer. Such properties include, for example, the ability to bind to the 8F5 and functionally-related antibodies described herein.

The terms “specific binding” or “specifically binding”, as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG3, IgG4, IgA1 and IgA2) or subclass.

The term “antigen-binding portion” of an antibody (or simply “antibody portion”), as used herein, refers to one or more fragments of an antibody that retain the ability to specifically bind to an antigen (e.g., Aβ(1-42) globulomer). It has been shown that the antigen-binding function of an antibody can be performed by one or more fragments of a full-length antibody. Such antibody embodiments may also be bispecific, dual specific, or multi-specific, specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., Intern. Appln. Public. No. WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed herein within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “antibody construct” as used herein refers to a polypeptide comprising one or more the antigen binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art and represented in Table 1.

TABLE 1
SEQUENCE OF HUMAN IgG HEAVY CHAIN CONSTANT
DOMAIN AND LIGHT CHAIN CONSTANT DOMAIN

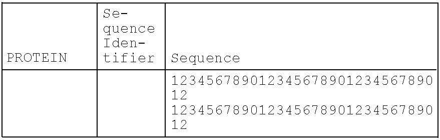

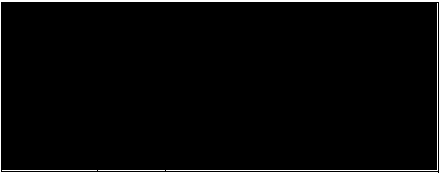

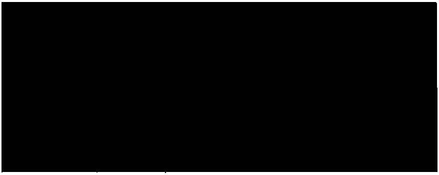

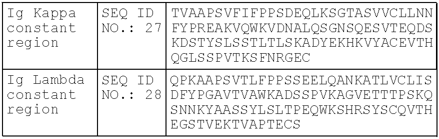

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody”, as used herein, is intended to refer to an antibody that is substantially free of other antibodies having different antigenic specificities (e.g., an isolated antibody that specifically binds Aβ(1-42) globulomer is substantially free of antibodies that specifically bind antigens other than Aβ(1-42) globulomer). An isolated antibody that specifically binds Aβ(1-42) globulomer may, however, have cross-reactivity to other antigens, such as Aβ(1-42) globulomer molecules from other species. Moreover, an isolated antibody may be substantially free of other cellular material and/or chemicals.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described below), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences.

The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, preferably, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In a preferred embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Human heavy chain and light chain acceptor sequences are known in the art. In one embodiment of the invention the human heavy chain and light chain acceptor sequences are selected from the sequences described below:

TABLE 2
HEAVY CHAIN ACCEPTOR SEQUENCES

| | | |
|---|---|---|
| SEQ | | |
| ID | Protein | |
| No. | region | Sequence |

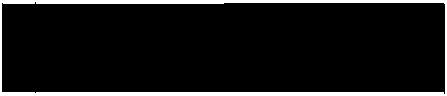

TABLE 3
LIGHT CHAIN ACCEPTOR SEQUENCES

| SEQ | | |
|---|---|---|
| ID | Protein | |
| No. | region | Sequence |
| 21 | A19/JK1 Fr1 | DIVMTQSPLSLPVTPGEPASISC |
| 21 | A19/JK1 Fr1 | DIVMTQSPLSLPVTPGEPASISC |
| 22 | A19/JK1 Fr2 | WYLQKPGQSPQLLIY |
| 23 | A19/JK1 Fr3 | GVPDRFSGSGSGTDFTLKISRVEAEDVGVYYC |
| 24 | A19/JK1 Fr4 | FGGGTKVEIKR |

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al., Crit. Rev. Immunol. 22(3): 183-200 (2002); Marchalonis et al., Adv Exp Med Biol. 484:13-30 (2001)). One of the advantages provided by various embodiments of the present invention stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

As used herein, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, preferably at least 85%, more preferably at least 90%, more preferably at least 95%, more preferably at least 98% and most preferably at least 99% identical to the amino acid sequence of a non-human antibody CDR. A humanized antibody comprises substantially all of at least one, and typically two, variable domains (Fab, Fab′, F(ab′) 2, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In other embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgM, IgG, IgD, IgA and IgE, and any isotype, including without limitation IgG 1, IgG2, IgG3 and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

As used herein, “Vernier” zone refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and may impact on the structure of CDRs and the affinity of the antibody.

As used herein, the term “neutralizing” refers to neutralization of biological activity of a globulomer when a binding protein specifically binds the globulomer. Preferably, a neutralizing binding protein is a neutralizing antibody whose binding to the Aβ(1-42) amino acid region of the globulomer results in inhibition of a biological activity of the globulomer. Preferably the neutralizing binding protein binds to the Aβ(1-42) region of the globulomer and reduces a biologically activity of the globulomer by at least about 20%, 40%, 60%, 80%, 85% or more. Inhibition of a biological activity of the globulomer by a neutralizing binding protein can be assessed by measuring one or more indicators of globulomer biological activity well known in the art.

The term “activity” includes activities such as the binding specificity/affinity of an antibody for an antigen, for example, an anti-Aβ(1-42) antibody that binds to an Aβ(1-42) globulomer and/or the neutralizing potency of an antibody, for example, an anti-Aβ(1-42) antibody whose binding to Aβ(1-42) inhibits the biological activity of the globulomer.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “Kₒₙ”, as used herein, is intended to refer to the on rate constant for association of an antibody to the antigen to form the antibody/antigen complex as is known in the art.

The term “K_{off}”, as used herein, is intended to refer to the off rate constant for dissociation of an antibody from the antibody/antigen complex as is known in the art.

The term “K_{d}” or “K_{D}”, as used herein, is intended to refer to the dissociation constant of a particular antibody-antigen interaction as is known in the art.

The term “labeled binding protein” as used herein, refers to a protein with a label incorporated that provides for the identification of the binding protein. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody conjugate” refers to a binding protein, such as an antibody, chemically linked to a second chemical moiety, such as a therapeutic or cytotoxic agent. The term “agent” is used herein to denote a chemical compound, a mixture of chemical compounds, a biological macromolecule, or an extract made from biological materials. Preferably the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

The terms “crystal”, and “crystallized” as used herein, refer to an antibody, or antigen-binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ed., pp. 20 1-16, Oxford University Press, New York, N.Y., (1999).”

The term “polynucleotide” as referred to herein means a polymeric form of two or more nucleotides, either ribonucleotides or deoxynucleotides or a modified form of either type of nucleotide. The term includes single and double stranded forms of DNA but preferably is double-stranded DNA.

The term “isolated polynucleotide” as used herein shall mean a polynucleotide (e.g., of genomic, cDNA, or synthetic origin, or some combination thereof) that, by virtue of its origin, is not associated with all or a portion of a polynucleotide with which the “isolated polynucleotide” is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells that transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E. coli; mammalian cell lines CHO, HEK 293 and COS; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

“Transgenic organism”, as known in the art and as used herein, refers to an organism having cells that contain a transgene, wherein the transgene introduced into the organism (or an ancestor of the organism) expresses a polypeptide not naturally expressed in the organism. A “transgene” is a DNA construct, which is stably and operably integrated into the genome of a cell from which a transgenic organism develops, directing the expression of an encoded gene product in one or more cell types or tissues of the transgenic organism.

The term “regulate” and “modulate” are used interchangeably, and, as used herein, refers to a change or an alteration in the activity of a molecule of interest (e.g., the biological activity of Aβ(1-42) globulomer). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator,” as used herein, is a compound capable of changing or altering an activity or function of a molecule of interest (e.g., the biological activity of Aβ(1-42) globulomer). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in International Application Publication No. WO 01/83525.

The term “agonist”, as used herein, refers to a modulator that, when contacted with a molecule of interest, causes an increase in the magnitude of a certain activity or function of the molecule compared to the magnitude of the activity or function observed in the absence of the agonist. Particular agonists of interest may include, but are not limited to, Aβ(1-42) globulomer polypeptides or polypeptides, nucleic acids, carbohydrates, or any other molecules that bind to Aβ(1-42) globulomer.

The term “antagonist” or “inhibitor”, as used herein, refer to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of Aβ(1-42) globulomer. Antagonists and inhibitors of Aβ(1-42) globulomer may include, but are not limited to, proteins, nucleic acids, carbohydrates, or any other molecules, which bind to Aβ(1-42) globulomer.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

The term “sample”, as used herein, is used in its broadest sense. A “biological sample”, as used herein, includes, but is not limited to, any quantity of a substance from a living thing or formerly living thing. Such living things include, but are not limited to, humans, mice, rats, monkeys, dogs, rabbits and other mammalian or non-mammalian animals. Such substances include, but are not limited to, blood, serum, urine, synovial fluid, cells, organs, tissues (e.g., brain), bone marrow, lymph nodes, cerebrospinal fluid, and spleen.

Antibodies that Bind Aβ(1-42) Globulomer

One aspect of the present invention provides isolated murine monoclonal antibodies, or antigen-binding portions thereof, that bind to Aβ(1-42) globulomer with high affinity, a slow off rate and high neutralizing capacity. A second aspect of the invention provides chimeric antibodies that bind Aβ(1-42) globulomer. A third aspect of the invention provides CDR grafted antibodies, or antigen-binding portions thereof, that bind Aβ(1-42) globulomer. A fourth aspect of the invention provides humanized antibodies, or antigen-binding portions thereof, that bind Aβ(1-42) globulomer. Preferably, the antibodies, or portions thereof, are isolated antibodies. Preferably, the antibodies of the invention are neutralizing human anti-Aβ(1-42) globulomer antibodies.

### A. Method of Making Anti-Aβ(1-42) Globulomer Antibodies

Antibodies of the present invention may be made by any of a number of techniques known in the art. Several of these methods are described in detail as follows:

### 1. Anti-Aβ(1-42) Globulomer Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In one embodiment, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention. Briefly, mice can be immunized with an Aβ(1-42) globulomer antigen. In a preferred embodiment, the antigen is administered with a adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

After immunization of an animal with an Aβ(1-42) globulomer antigen, antibodies and/or antibody-producing cells may be obtained from the animal. An anti-Aβ(1-42) globulomer antibody-containing serum is obtained from the animal by bleeding or sacrificing the animal. The serum may be used as it is obtained from the animal, an immunoglobulin fraction may be obtained from the serum, or the anti-Aβ(1-42) globulomer antibodies may be purified from the serum. Serum or immunoglobulins obtained in this manner are polyclonal, thus having a heterogeneous array of properties.

Once an immune response is detected, e.g., antibodies specific for the antigen Aβ(1-42) globulomer are detected in the mouse serum, the mouse spleen is harvested and splenocytes isolated. The splenocytes are then fused by well-known techniques to any suitable myeloma cells, for example cells from cell line SP20 available from the American Type Culture Collection (Manassas, Va.). Hybridomas are selected and cloned by limited dilution. The hybridoma clones are then assayed by methods known in the art for cells that secrete antibodies capable of binding Aβ(1-42) globulomer. Ascites fluid, which generally contains high levels of antibodies, can be generated by immunizing mice with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using Aβ(1-42) globulomer, or a portion thereof, or a cell expressing Aβ(1-42) globulomer. In a preferred embodiment, the initial screening is performed using an enzyme-linked immunoassay (ELISA) or a radioimmunoassay (RIA), preferably an ELISA. An example of ELISA screening is provided in International Application Publication No. WO 00/37504, herein incorporated by reference.

Anti-Aβ(1-42) globulomer antibody-producing hybridomas are selected, cloned and further screened for desirable characteristics, including robust hybridoma growth, high antibody production and desirable antibody characteristics, as discussed further below. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, the hybridomas are mouse hybridomas, as described above. In another preferred embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an anti-Aβ(1-42) globulomer antibody.

Antibody fragments that recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

### 2. Anti-Aβ(1-42) Globulomer Monoclonal Antibodies Using Slam

In another aspect of the invention, recombinant antibodies are generated from single, isolated lymphocytes using a procedure referred to in the art as the selected lymphocyte antibody method (SLAM), as described in U.S. Pat. No. 5,627,052, International Application Publication No. WO 92/02551 and Babcock, J. S. et al. (1996) Proc. Natl. Acad. Sci. USA 93:7843-7848. In this method, single cells secreting antibodies of interest, e.g., lymphocytes derived from any one of the immunized animals described in Section 1, are screened using an antigen-specific hemolytic plaque assay, wherein the antigen Aβ(1-42) globulomer, a subunit of Aβ(1-42) globulomer, or a fragment thereof, is coupled to sheep red blood cells using a linker, such as biotin, and used to identify single cells that secrete antibodies with specificity for Aβ(1-42) globulomer. Following identification of antibody-secreting cells of interest, heavy- and light-chain variable region cDNAs are rescued from the cells by reverse transcriptase-PCR and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example by panning the transfected cells to isolate cells expressing antibodies to Aβ(1-42) globulomer. The amplified immunoglobulin sequences further can be manipulated in vitro, such as by in vitro affinity maturation methods such as those described in International Application Publication No. WO 97/29131 and International Application Publication No. WO 00/56772.

### 3. Anti-Aβ(1-42) Globulomer Monoclonal Antibodies Using Transgenic Animals

In another embodiment of the instant invention, antibodies are produced by immunizing a non-human animal comprising some, or all, of the human immunoglobulin locus with an Aβ(1-42) globulomer antigen. In a preferred embodiment, the non-human animal is a XENOMOUSE transgenic mouse, an engineered mouse strain that comprises large fragments of the human immunoglobulin loci and is deficient in mouse antibody production. See, e.g., Green et al. Nature Genetics 7:13-21 (1994) and U.S. Pat. Nos. 5,916,771, 5,939,598, 5,985,615, 5,998,209, 6,075,181, 6,091,001, 6,114,598 and 6,130,364. See also Internation Appln. Publication No. WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00/09560, published Feb. 24, 2000 and WO 00/037504, published Jun. 29, 2000. The XENOMOUSE transgenic mouse produces an adult-like human repertoire of fully human antibodies and generates antigen-specific human Mabs. The XENOMOUSE transgenic mouse contains approximately 80% of the human antibody repertoire through introduction of megabase sized, germline configuration YAC fragments of the human heavy chain loci and x light chain loci. See Mendez et al., Nature Genetics 15:146-156 (1997), Green and Jakobovits J. Exp. Med. 188:483-495 (1998), the disclosures of which are hereby incorporated by reference.

### 4. Anti-Aβ(1-42) Globulomer Monoclonal Antibodies Using Recombinant Antibody Libraries

In vitro methods also can be used to make the antibodies of the invention, wherein an antibody library is screened to identify an antibody having the desired binding specificity. Methods for such screening of recombinant antibody libraries are well known in the art and include methods described in, for example, Ladner et al., U.S. Pat. No. 5,223,409; Kang et al., International Appln. Publication No. WO 92/18619; Dower et al., International Appln. Publication No. WO 91/17271; Winter et al., International Appln. Publication No. WO 92/20791; Markland et al., International Appln. Publication No. WO 92/15679; Breitling et al., International Appln. Publication No. WO 93/01288; McCafferty et al., PCT Publication No. WO 92/01047; Garrard et al., International Appln. Publication No. WO 92/09690; Fuchs et al. (1991), Bio/Technology 9:1370-1372; Hay et al., (1992) Hum Antibod Hybridomas 3:81-85; Huse et al. (1989), Science 246:1275-1281; McCafferty et al., Nature (1990) 348:552-554; Griffiths et al. (1993) EMBO J 12:725-734; Hawkins et al., (1992) J Mol Biol 226:889-896; Clackson et al., (1991) Nature 352:624-628; Gram et al., (1992) PNAS 89:3576-3580; Garrad et al. (1991) Bio/Technology 9:1373-1377; Hoogenboom et al. (1991), Nuc Acid Res 19:4133-4137; and Barbas et al. (1991), PNAS 88:7978-7982, U.S. Patent Application Publication No. 20030186374, and International Application Publication No. WO 97/29131, the contents of each of which are incorporated herein by reference.

The recombinant antibody library may be from a subject immunized with Aβ(1-42) globulomer, or a portion of Aβ(1-42) globulomer. Alternatively, the recombinant antibody library may be from a naïve subject, i.e., one who has not been immunized with Aβ(1-42) globulomer, such as a human antibody library from a human subject who has not been immunized with human Aβ(1-42) globulomer. Antibodies of the invention are selected by screening the recombinant antibody library with the peptide comprising human Aβ(1-42) globulomer to thereby select those antibodies that recognize Aβ(1-42) globulomer. Methods for conducting such screening and selection are well known in the art, such as described in the references in the preceding paragraph. To select antibodies of the invention having particular binding affinities for Aβ(1-42) globulomer, such as those that dissociate from human Aβ(1-42) globulomer with a particular k_{off} rate constant, the art-known method of surface plasmon resonance can be used to select antibodies having the desired k_{off} rate constant. To select antibodies of the invention having a particular neutralizing activity for Aβ(1-42) globulomer, such as those with a particular IC₅₀, standard methods known in the art for assessing the inhibition of human Aβ(1-42) globulomer activity may be used.

In one aspect, the invention pertains to an isolated antibody, or an antigen-binding portion thereof, that binds human Aβ(1-42) globulomer. Preferably, the antibody is a neutralizing antibody. In various embodiments, the antibody is a recombinant antibody or a monoclonal antibody.

For example, the antibodies of the present invention can also be generated using various phage display methods known in the art. In phage display methods, functional antibody domains are displayed on the surface of phage particles that carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); International Application No. PCT/GB91/01134; International Appln. Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, each of which is incorporated herein by reference in its entirety.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in International Application Publ. No. WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

Alternative to screening of recombinant antibody libraries by phage display, other methodologies known in the art for screening large combinatorial libraries can be applied to the identification of dual specificity antibodies of the invention. One type of alternative expression system is one in which the recombinant antibody library is expressed as RNA-protein fusions, as described in International Appln. Publication No. WO 98/31700 by Szostak and Roberts, and in Roberts, R. W. and Szostak, J. W. (1997) Proc. Natl. Acad. Sci. USA 94:12297-12302. In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above.

In another approach the antibodies of the present invention can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies of the present invention include those disclosed Wittrup et al., U.S. Pat. No. 6,699,658 incorporated herein by reference.

### B. Production of Recombinant Aβ(1-42) Globulomer Antibodies

As noted above, antibodies of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies of the invention in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of an antibody of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the antigens of interest by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the invention provides a method of synthesizing a recombinant antibody of the invention by culturing a host cell of the invention in a suitable culture medium until a recombinant antibody of the invention is synthesized. The method can further comprise isolating the recombinant antibody from the culture medium.

### 1. Anti-Aβ(1-42) Globulomer Antibodies

The isolated anti-Aβ(1-42) globulomer antibody CDR sequences described herein (see Table 4) establish a novel family of Aβ(1-42) globulomer binding proteins, isolated in accordance with this invention, and comprising polypeptides that include the CDR sequences listed above. To generate and to select CDRs of the invention having preferred Aβ(1-42) globulomer binding and/or neutralizing activity with respect to Aβ(1-42) globulomer, standard methods known in the art for generating binding proteins of the present invention and assessing the Aβ(1-42) globulomer binding and/or neutralizing characteristics of those binding protein may be used, including but not limited to those specifically described herein.

### 2. Anti-Aβ(1-42) Globulomer Chimeric Antibodies

A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art and discussed in detail in Example 2.1. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entireties. In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454 which are incorporated herein by reference in their entireties) by splicing genes from a mouse antibody molecule of appropriate antigen specificity together with genes from a human antibody molecule of appropriate biological activity can be used.

In one embodiment, the chimeric antibodies of the invention are produced by replacing the heavy chain constant region of the murine monoclonal anti-human Aβ(1-42) globulomer antibodies described above with a human IgG1 constant region. In a specific embodiment, the chimeric antibody of the invention comprises a heavy chain variable region (V_{H}) comprising the amino acid sequence of SEQ ID NO:1 and a light chain variable region (V_{L}) comprising the amino acid sequence of SEQ ID NO:2.

### 3. Anti-Aβ(1-42) Globulomer CDR Grafted Antibodies

CDR-grafted antibodies of the invention comprise heavy and light chain variable region sequences from a human antibody wherein one or more of the CDR regions of V_{H} and/or V_{L} are replaced with CDR sequences of the murine antibodies of the invention. A framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, it is preferable that the human variable framework that is chosen to replace the murine variable framework apart from the CDRs have at least a 65% sequence identity with the murine antibody variable region framework. It is more preferable that the human and murine variable regions apart from the CDRs have at least 70% sequence identify. It is even more preferable that the human and murine variable regions apart from the CDRs have at least 75% sequence identity. It is most preferable that the human and murine variable regions apart from the CDRs have at least 80% sequence identity. Methods for producing chimeric antibodies are known in the art and discussed in detail in Example 2.2. (See also EP 239,400; Intern. Appln. Publication No. WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,352).

### 4. Anti-Aβ(1-42) Globulomer Humanized Antibodies

Table 4 below includes a list of amino acid sequences of VH and VL regions of preferred anti-Aβ(1-42) humanized globulomer antibodies of the invention as well as the CDRs contained therein.

TABLE 4
LIST OF AMINO ACID SEQUENCES OF VH AND VL REGIONS

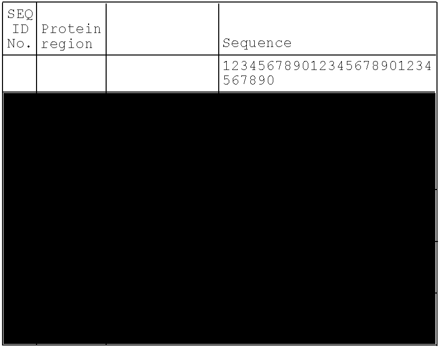

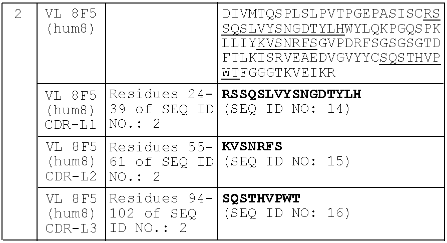

Humanized antibodies are antibody molecules from non-human species antibody that bind the desired antigen having one or more complementarity determining regions (CDRs) from the non-human species and framework regions from a human immunoglobulin molecule. Known human Ig sequences are disclosed, e.g., www.ncbi.nlm.nih.gov/entrez-/query.fcgi; www.atcc.org/phage/hdb.html; www.sciquest.com/; www.abcam.com/; www.antibodyresource.com/onlinecomp.html; www.public.iastate.edu/.about.pedro/research_tools.html; www.mgen.uni-heidelberg.de/SD/IT/IT.html; www.whfreeman.com/immunology/CH-05/kubyO5.htm; www.library.thinkquest.org/12429/Immune/Antibody.html; www.hhmi.org/grants/lectures/1996/vlab/; www.path.cam.ac.uk/.about.mrc7/m-ikeimages.html; www.antibodyresource.com/; mcb.harvard.edu/BioLinks/Immunology.html.www.immunologylink.com/; pathbox.wustl.edu/.about.hcenter/index.-html; www.biotech.ufl.edu/.about.hcl/; www.pebio.com/pa/340913/340913.html-; www.nal.usda.gov/awic/pubs/antibody/; www.m.ehimeu.acjp/.about.yasuhito-/Elisa.html; www.biodesign.com/table.asp; www.icnet.uk/axp/facs/davies/lin-ks.html; www.biotech.ufl.edu/.about.fccl/protocol.html; www.isac-net.org/sites_geo.html; aximtl.imt.unimarburg.de/.about.rek/AEP-Start.html; baserv.uci.kun.nl/.about.jraats/linksl.html; www.recab.unihd.de/immuno.bme.nwu.edu/; www.mrc-cpe.cam.ac.uk/imt-doc/public/INTRO.html; www.ibt.unam.mx/vir/V_mice.html; imgt.cnusc.fr:8104/; www.biochem.ucl.ac.uk/.about.martin/abs/index.html; antibody.bath.ac.uk/; abgen.cvm.tamu.edu/lab/wwwabgen.html; www.unizh.ch/.about.honegger/AHOsem-inar/Slide01.html; www.cryst.bbk.ac.uk/.about.ubcg07s/; www.nimr.mrc.ac.uk/CC/ccaewg/ccaewg.htm; www.path.cam.ac.uk/.about.mrc7/h-umanisation/TAHHP.html; www.ibt.unam.mx/vir/structure/stat_aim.html; www.biosci.missouri.edu/smithgp/index.html; www.cryst.bioc.cam.ac.uk/.abo-ut.fmolina/Webpages/Pept/spottech.html; www.jerini.de/frroducts.htm; www.patents.ibm.com/ibm.html.Kabat et al., Sequences of Proteins of Immunological Interest, U.S. Dept. Health (1983), each entirely incorporated herein by reference. Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, such as but not limited to those described in Jones et al., Nature 321:522 (1986); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28(4/5):489-498 (1991); Studnicka et al., Protein Engineering 7(6):805-814 (1994); Roguska et al., PNAS 91:969-973 (1994); International Appln. Publication No. WO 91/09967, PCT/: US98/16280, US96/18978, US91/09630, US91/05939, US94/01234, GB89/01334, GB91/01134, GB92/01755; WO90/14443, WO90/14424, WO90/14430, EP 229246, EP 592,106; EP 519,596, EP 239,400, U.S. Pat. Nos. 5,565,332, 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, each entirely incorporated herein by reference, included references cited therein.

### C. Production of Antibodies and Antibody-Producing Cell Lines

As noted above, preferably, anti-Aβ(1-42) globulomer antibodies of the present invention exhibit a high capacity to reduce or to neutralize Aβ(1-42) globulomer activity, e.g., as assessed by any one of several in vitro and in vivo assays known in the art (e.g., see Examples below).

In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

Replacements of amino acid residues in the Fc portion to alter antibody effector function are known in the art (Winter et al., U.S. Pat. Nos. 5,648,260 and 5,624,821). The Fc portion of an antibody mediates several important effector functions, for example, cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. Neonatal Fc receptors (FcRn) are the critical components determining the circulating half-life of antibodies. In still another embodiment, at least one amino acid residue is replaced in the constant region of the antibody, for example the Fc region of the antibody, such that effector functions of the antibody are altered.

One embodiment provides a labeled binding protein wherein an antibody or antibody portion of the invention is derivatized or linked to another functional molecule (e.g., another peptide or protein). For example, a labeled binding protein of the invention can be derived by functionally linking an antibody or antibody portion of the invention (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

Another embodiment of the invention provides a crystallized binding protein. Preferably, the invention relates to crystals of whole anti-Aβ(1-42) globulomer antibodies and fragments thereof as disclosed herein, and formulations and compositions comprising such crystals. In one embodiment the crystallized binding protein has a greater half-life in vivo than the soluble counterpart of the binding protein. In another embodiment, the binding protein retains biological activity after crystallization.

Crystallized binding protein of the invention may be produced according methods known in the art and as disclosed in International Appln. Publication No. WO 02/072636, incorporated herein by reference.

Another embodiment of the invention provides a glycosylated binding protein wherein the antibody or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Antibodies are glycoproteins with one or more carbohydrate residues in the Fc domain, as well as the variable domain. Carbohydrate residues in the Fc domain have important effect on the effector function of the Fc domain, with minimal effect on antigen binding or half-life of the antibody (R. Jefferis, Biotechnol. Prog. 21 (2005), pp. 11-16). In contrast, glycosylation of the variable domain may have an effect on the antigen binding activity of the antibody. Glycosylation in the variable domain may have a negative effect on antibody binding affinity, likely due to steric hindrance (Co, M. S., et al., Mol. Immunol. (1993) 30:1361-1367), or result in increased affinity for the antigen (Wallick, S. C., et al., Exp. Med. (1988) 168:1099-1109; Wright, A., et al., EMBO J. (1991) 10:2717 2723).

One aspect of the present invention is directed to generating glycosylation site mutants in which the O- or N-linked glycosylation site of the binding protein has been mutated. One skilled in the art can generate such mutants using standard well-known technologies. The creation of glycosylation site mutants that retain the biological activity but have increased or decreased binding activity are another object of the present invention.

In still another embodiment, the glycosylation of the antibody or antigen-binding portion of the invention is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation may increase the affinity of the antibody for antigen. Such an approach is described in further detail in International Appln. Publication No. WO 03/016466A2, and U.S. Pat. Nos. 5,714,350 and 6,350,861, each of which is incorporated herein by reference in its entirety.

Additionally or alternatively, a modified antibody of the invention can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; International Appln. Publication Nos. WO 03/035835 and WO 99/5434280, each of which is incorporated herein by reference in its entirety.

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the invention may include, but are not limited to, glucose, galactose, mannose, fucose, n-acetylglucosamine and sialic acid. Preferably the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate antibodies or antigen-binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S Patent Application Publication Nos. 20040018590 and 20020137134 and International Appln. Publication No. WO 05/100584 A2).

The term “multivalent binding protein” is used in this specification to denote a binding protein comprising two or more antigen binding sites. The multivalent binding protein is preferably engineered to have the three or more antigen binding sites, and is generally not a naturally occurring antibody. The term “multispecific binding protein” refers to a binding protein capable of binding two or more related or unrelated targets. Dual variable domain (DVD) binding proteins as used herein, are binding proteins that comprise two or more antigen binding sites and are tetravalent or multivalent binding proteins. Such DVDs may be monospecific, i.e, capable of binding one antigen or multispecific, i.e., capable of binding two or more antigens. DVD binding proteins comprising two heavy chain DVD polypeptides and two light chain DVD polypeptides are referred to a DVD Ig. Each half of a DVD Ig comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. DVD binding proteins and methods of making DVD binding proteins are disclosed in U.S. patent application Ser. No. 11/507,050 and incorporated herein by reference.

One aspect of the invention pertains to a DVD binding protein comprising binding proteins capable of binding to Aβ(1-42) globulomer. Preferably, the DVD binding protein is capable of binding Aβ(1-42) globulomer and a second target.

In addition to the binding proteins, the present invention is also directed to an anti-idiotypic (anti-Id) antibody specific for such binding proteins of the invention. An anti-Id antibody is an antibody, which recognizes unique determinants generally associated with the antigen-binding region of another antibody. The anti-Id can be prepared by immunizing an animal with the binding protein or a CDR containing region thereof. The immunized animal will recognize, and respond to the idiotypic determinants of the immunizing antibody and produce an anti-Id antibody. The anti-Id antibody may also be used as an “immunogen” to induce an immune response in yet another animal, producing a so-called anti-anti-Id antibody.

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. Preferably, the protein having a particularly selected novel glycosylation pattern exhibits improved or altered biological properties.

### D. Uses of Anti-Aβ(1-42) Antibodies

Given their ability to bind to Aβ(1-42) globulomer, the anti-Aβ(1-42) globulomer antibodies, or portions thereof, of the invention can be used to detect Aβ(1-42) globulomer (e.g., in a biological sample such as serum, whole blood, CSF, brain tissue or plasma), using a conventional immunoassay, such as an enzyme linked immunosorbent assays (ELISA), an radioimmunoassay (RIA) or tissue immunohistochemistry. The invention therefore provides a method for detecting Aβ(1-42) globulomer in a biological sample comprising contacting a biological sample with an antibody, or antibody portion, of the invention and detecting either the antibody (or antibody portion) bound to Aβ(1-42) globulomer or unbound antibody (or antibody portion), to thereby detect Aβ(1-42) globulomer in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

Alternative to labeling the antibody, Aβ(1-42) globulomer can be assayed in biological fluids by a competition immunoassay utilizing recombinant Aβ(1-42) globulomer standards labeled with a detectable substance and an unlabeled anti-Aβ(1-42) globulomer antibody. In this assay, the biological sample, the labeled recombinant Aβ(1-42) globulomer standards and the anti-Aβ(1-42) globulomer antibody are combined, and the amount of labeled recombinant Aβ(1-42) globulomer standard bound to the unlabeled antibody is determined. The amount of Aβ(1-42) globulomer in the biological sample is inversely proportional to the amount of labeled rAβ(1-42) globulomer standard bound to the anti-Aβ(1-42) globulomer antibody.

The antibodies and antibody portions of the invention preferably are capable of neutralizing Aβ(1-42) globulomer activity both in vitro and in vivo. Accordingly, such antibodies and antibody portions of the invention can be used to inhibit Aβ(1-42) globulomer activity, e.g., in a cell culture containing Aβ(1-42) globulomer, in human subjects, or in other mammalian subjects having Aβ(1-42) globulomer with which an antibody of the invention cross-reacts. In one embodiment, the invention provides a method for inhibiting Aβ(1-42) globulomer activity comprising contacting Aβ(1-42) globulomer with an antibody or antibody portion of the invention such that Aβ(20-42) globulomer activity is inhibited. For example, in a cell culture containing, or suspected of containing Aβ(1-42) globulomer, an antibody or antibody portion of the invention can be added to the culture medium to inhibit Aβ(1-42) globulomer activity in the culture.

In another embodiment, the invention provides a method for reducing Aβ(1-42) globulomer activity in a subject, advantageously from a subject suffering from a disease or disorder in which Aβ(1-42) globulomer activity is detrimental (e.g., an amyloidosis such as Alzheimer's Disease). The invention therefore provides methods for reducing Aβ(1-42) globulomer activity in a subject suffering from such a disease or disorder, which method comprises administering to the subject an antibody or antibody portion of the invention such that Aβ(1-42) globulomer activity in the subject is reduced. Preferably, the Aβ(1-42) globulomer is human Aβ(1-42) globulomer, and the subject is a human subject. Alternatively, the subject can be a mammal expressing an Aβ(1-42) globulomer to which an antibody of the invention is capable of binding. Still further, the subject can be a mammal into which Aβ(1-42) globulomer has been introduced (e.g., by administration of Aβ(1-42) globulomer or by expression of Aβ(1-42) globulomer transgene). An antibody of the invention can be administered to a human subject for therapeutic purposes. Moreover, an antibody of the invention can be administered to a non-human mammal expressing Aβ(1-42) globulomer with which the antibody is capable of binding for veterinary purposes or as an animal model of human disease. Regarding the latter, such animal models may be useful for evaluating the therapeutic efficacy of antibodies of the invention (e.g., testing of dosages and time courses of administration).

As used herein, the term “a disorder in which Aβ(1-42) globulomer activity is detrimental” is intended to include diseases and other disorders in which the presence of Aβ(1-42) globulomer in a subject suffering from the disorder has been shown to be or is suspected of being either responsible for the pathophysiology of the disorder or a factor that contributes to a worsening of the disorder. Accordingly, a disorder in which Aβ(1-42) globulomer activity is detrimental is a disorder in which reduction of Aβ(1-42) globulomer activity is expected to alleviate some or all of the symptoms and/or progression of the disorder. Such disorders may be evidenced, for example, by an increase in the concentration of Aβ(1-42) globulomer in a biological fluid of a subject suffering from the disorder (e.g., an increase in the concentration of Aβ(1-42) globulomer in serum, brain tissue, plasma, cerebrospinal fluid, etc. of the subject), which can be detected, for example, using an anti-Aβ(1-42) globulomer antibody as described above. Non-limiting examples of disorders that can be treated with the antibodies of the invention include those disorders discussed in the section below pertaining to pharmaceutical compositions of the antibodies of the invention.

### D. Pharmaceutical Composition

The invention also provides pharmaceutical compositions comprising an antibody, or antigen-binding portion thereof, of the invention and a pharmaceutically acceptable carrier. The pharmaceutical compositions comprising antibodies of the invention are for use in, but not limited to, diagnosing, detecting, or monitoring a disorder, in preventing, treating, managing, or ameliorating of a disorder or one or more symptoms thereof, and/or in research. In a specific embodiment, a composition comprises one or more antibodies of the invention. In another embodiment, the pharmaceutical composition comprises one or more antibodies of the invention and one or more prophylactic or therapeutic agents other than antibodies of the invention for treating a disorder in which Aβ(1-42) globulomer activity is detrimental. Preferably, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The antibodies and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

Various delivery systems are known and can be used to administer one or more antibodies of the invention or the combination of one or more antibodies of the invention and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and International Appln. Publication Nos. WO 92/19244, WO 97/32572, WO 97/44013, WO 98/31346, and WO 99/66903, each of which is incorporated herein by reference their entireties. In one embodiment, an antibody of the invention, combination therapy, or a composition of the invention is administered using Alkermes AIR® pulmonary drug delivery technology (Alkermes, Inc., Cambridge, Mass.). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies of the invention antagonists is administered locally to the affected area to a subject to prevent, treat, manage, and/or ameliorate a disorder or a symptom thereof. In another embodiment, an effective amount of one or more antibodies of the invention is administered locally to the affected area in combination with an effective amount of one or more therapies (e.g., one or more prophylactic or therapeutic agents) other than an antibody of the invention of a subject to prevent, treat, manage, and/or ameliorate a disorder or one or more symptoms thereof.

In another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; International Appln. Publication No. WO 99/15154; and International Appln. Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, International Appln. Publication No. WO 91/05548, International Appln. Publication No. WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entireties.

In a specific embodiment, where the composition of the invention is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lidocaine to ease pain at the site of the injection.

If the compositions of the invention are to be administered topically, the compositions can be formulated in the form of an ointment, cream, transdermal patch, lotion, gel, shampoo, spray, aerosol, solution, emulsion, or other form well known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol, the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the invention may comprise pulmonary administration, e.g., by use of an inhaler or nebulizer, of a composition formulated with an aerosolizing agent. See, e.g., U.S. Pat. Nos. 6,019,968, 5,985,320, 5,985,309, 5,934,272, 5,874,064, 5,855,913, 5,290,540, and 4,880,078; and International Appln. Publication Nos. WO 92/19244, WO 97/32572, WO 97/44013, WO 98/31346, and WO 99/66903, each of which is incorporated herein by reference their entireties. In a specific embodiment, an antibody of the invention, combination therapy, and/or composition of the invention is administered using Alkermes AIR® pulmonary drug delivery technology (Alkermes, Inc., Cambridge, Mass.).

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use. The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the invention encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

The antibodies and antibody portions of the invention can be incorporated into a pharmaceutical composition suitable for parenteral administration. Preferably, the antibody or antibody portions will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants. The pharmaceutical composition comprising the antibodies and antibody-portions of the invention prepared as an injectable solution for parenteral administration, can further comprise an agent useful as an adjuvant, such as those used to increase the absorption, or dispersion of a therapeutic protein (e.g., antibody). A particularly useful adjuvant is hyaluronidase, such as Hylenex® (recombinant human hyaluronidase). Addition of hyaluronidase in the injectable solution improves human bioavailability following parenteral administration, particularly subcutaneous administration. It also allows for greater injection site volumes (i.e. greater than 1 ml) with less pain and discomfort, and minimum incidence of injection site reactions. (See International Appln. Publication No. WO 04/078140 and U.S. Patent Appln. Publication No. US2006104968, incorporated herein by reference.)

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The antibodies and antibody portions of the present invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion of the invention is coformulated with and/or coadministered with one or more additional therapeutic agents that are useful for treating disorders in which Aβ(1-42) activity is detrimental. For example, an anti-Aβ(1-42) antibody or antibody portion of the invention may be coformulated and/or coadministered with one or more additional antibodies that bind other targets (e.g., antibodies that bind other cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, an antibody to Aβ(1-42) or fragment thereof is linked to a half-life extending vehicle known in the art. Such vehicles include, but are not limited to, the Fc domain, polyethylene glycol, and dextran. Such vehicles are described, e.g., in U.S. patent application Ser. No. 09/428,082 and published International Patent Application No. WO 99/25044, which are hereby incorporated by reference for any purpose.

In a specific embodiment, nucleic acid sequences comprising nucleotide sequences encoding an antibody of the invention or another prophylactic or therapeutic agent of the invention are administered to treat, prevent, manage, or ameliorate a disorder or one or more symptoms thereof by way of gene therapy. Gene therapy refers to therapy performed by the administration to a subject of an expressed or expressible nucleic acid. In this embodiment of the invention, the nucleic acids produce their encoded antibody or prophylactic or therapeutic agent of the invention that mediates a prophylactic or therapeutic effect.

Any of the methods for gene therapy available in the art can be used according to the present invention. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley &Sons, NY (1993); and Kriegler, Gene Transfer and Expression, A Laboratory Manual, Stockton Press, NY (1990). Detailed description of various methods of gene therapy are disclosed in U.S. Patent Application Publication No. US20050042664 A1 which is incorporated herein by reference. Antibodies of the invention or antigen binding portions thereof can be used alone or in combination to treat diseases such as Alpha1-antitrypsin-deficiency, C1-inhibitor deficiency angioedema, Antithrombin deficiency thromboembolic disease, Kuru, Creutzfeld-Jacob disease/scrapie, Bovine spongiform encephalopathy, Gerstmann-Straussler-Scheinker disease, Fatal familial insomnia, Huntington's disease, Spinocerebellar ataxia, Machado-Joseph atrophy, Dentato-rubro-pallidoluysian atrophy, Frontotemporal dementia, Sickle cell anemia, Unstable hemoglobin inclusion-body hemolysis, Drug-induced inclusion body hemolysis, Parkinson's disease, Systemic AL amyloidosis, Nodular AL amyloidosis, Systemic AA amyloidosis, Prostatic amyloid, Hemodialysis amyloidosis, Hereditary (Icelandic) cerebral angiopathy, Huntington's disease, Familial visceral amyloid, Familial visceral polyneuropathy, Familial visceral amyloidosis, Senile systemic amyloidosis, Familial amyloid neurophathy, Familial cardiac amyloid, Alzheimer's disease, Down's syndrome, Medullary carcinoma thyroid and Type 2 diabetes mellitus (T2DM). Preferably, the antibodies of the present invention may be utilized to treat an amyloidosis, for example, Alzheimer's disease and Down's syndrome.

It should be understood that the antibodies of the invention or antigen binding portion thereof can be used alone or in combination with one or more additional agents, e.g., a therapeutic agent (for example, a small molecule or biologic), said additional agent being selected by the skilled artisan for its intended purpose. For example, the additional agent can be a therapeutic agent such as a cholesterinase inhibitor (e.g., tactrine, donepezil, rivastigmine or galantamine), a partial NMDA receptor blocker (e.g., memantine), a glycosaminoglycan mimetic (e.g., Alzhemed), an inhibitor or allosteric modulator of gamma secretase (e.g., R-flurbiprofen), a luteinizing hormone blockade gonadotropin releasing hormone agonist (e.g., leuprorelin), a serotinin 5-HT1A receptor antagonist, a chelatin agent, a neuronal selective L-type calcium channel blocker, an immunomodulator, an amyloid fibrillogenesis inhibitor or amyloid protein deposition inhibitor (e.g., M266), another antibody (e.g., bapineuzumab), a 5-HT1a receptor antagonist, a PDE4 inhibitor, a histamine agonist, a receptor protein for advanced glycation end products, a PARP stimulator, a serotonin 6 receptor antagonist, a 5-HT4 receptor agonist, a human steroid, a glucose uptake stimulant which enhanceds neuronal metabolism, a selective CB1 antagonist, a partial agonist at benzodiazepine receptors, an amyloid beta production antagonist or inhibitor, an amyloid beta deposition inhibitor, a NNR alpha-7 partial antagonist, a cytokine inhibitor, a TNF antagonist (e.g., Humira and Remicade), a TNF receptor fusion protein (e.g., Enbrel), a therapeutic targeting PDE4, a RNA translation inhibitor, a muscarinic agonist, a nerve growth factor receptor agonist, a NGF receptor agonist and a gene therapy modulator (i.e., those agents currently recognized, or in the future being recognized, as useful to treat the disease or condition being treated by the antibody of the present invention). The additional agent also can be an agent that imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations, which are part of this invention, can be the antibodies of the present invention and at least one additional agent selected from the lists below. The combination can also include more than one additional agent, e.g., two or three additional agents if the combination is such that the formed composition can perform its intended function.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may be determined by a person skilled in the art and may vary according to factors such as the disease state, age, sex, and weight of the individual, and the ability of the antibody or antibody portion to elicit a desired response in the individual. A therapeutically effective amount is also one in which any toxic or detrimental effects of the antibody, or antibody portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

It will be readily apparent to those skilled in the art that other suitable modifications and adaptations of the methods of the invention described herein are obvious and may be made using suitable equivalents without departing from the scope of the invention or the embodiments disclosed herein. Having now described the present invention in detail, the same will be more clearly understood by reference to the following examples, which are included for purposes of illustration only and are not intended to be limiting of the invention.

### Example I

### Generation and Isolation of Humanized Anti-Aβ(1-42)

### Globulomer Monoclonal Antibodies

This example describes the humanization of an anti-A-beta antibody. Humanization of the murine monoclonal antibody 8F5 (Mu8F5) was carried out essentially according to the procedure of Queen, C., et al., Proc. Natl. Acad. Sci. USA 86: 10029-10033 (1989). First, human V segments with high homology to the Mu8F5 VH or VL amino acid sequences were identified. Next, the complementarity-determining region (CDR) sequences together with framework amino acids important for maintaining the structures of the CDRs were grafted into the selected human framework sequences. In addition, human framework amino acids that were found to be rare in the corresponding V region subgroup were substituted with consensus amino acids to reduce potential immunogenicity. The resulting humanized monoclonal antibody (Hu8F5) was expressed in the human kidney cell line 293T/17. Using a competitive binding assay with purified 8F5 antibodies, the affinity of Hu8F5 to human A-beta was shown to be equivalent to that of Mu8F5.

### Materials and Methods

### Humanization:

Humanization of the antibody V regions was carried out as outlined by Queen, C., et al., ibid. The human V region frameworks used as acceptors for the CDRs of Mu8F5 were chosen based on sequence homology. The computer programs ABMOD and ENCAD (Levitt, M., J. Mol. Biol. 168: 595-620 (1983)) were used to construct a molecular model of the variable regions. Amino acids in the humanized V regions predicted to have contact with the CDRs were substituted with the corresponding residues of Mu8F5. Amino acids in the humanized V region that were found to be rare in the same V region subgroup were changed to consensus amino acids to eliminate potential immunogenicity.

The heavy and light chain variable region genes were designed using approximately 30 overlapping synthetic oligonucleotides ranging in length from approximately 20 to 40 bases following a published method (Rouillard, J.-M., et al., Nucleic Acids Res. 32: W176-W180 (2004)). The oligonucleotides were annealed and assembled with PfuTurbo DNA Polymerase (Stratagene, La Jolla, Calif.), yielding a full-length product. The resulting product was amplified by the polymerase chain reaction (PCR) using PfuTurbo DNA Polymerase (Stratagene). The PCR-amplified fragments were gel-purified, and cloned into the pCR4Blunt-TOPO vector (Invitrogen Corporation, Carlsbad, Calif.). After sequence confirmation, Hu8F5 VH and Hu8F5 VL were digested with MluI and XbaI, gel-purified, and subcloned, respectively, into a modified form of pVg1.D.Tt (Cole, M. S., et al., J. Immunol. 159: 3613-3621 (1997); and see below) and pVk (Co, M. S., et al., J. Immunol. 148: 1149-1154 (1992)). The final plasmids were verified by restriction mapping. The sequences of the variable regions of the heavy and light chains were verified by nucleotide sequencing.

### Site-Directed Mutagenesis

Site-directed mutagenesis of the synthetic V-genes was done using the QuikChange II Site-Directed Mutagenesis Kit (Stratagene), following the manufacturer's recommendations. To generate the W47L mutation in the Hu8F5VH gene sequence, a pair of synthetic oligonucleotide primers both containing the desired mutation was designed. The mutagenesis primers 5′W47L (5′-CTG GCA AGG GCC TGG AGC TGG TGG CCA GCA TCA ACA GCA AC-3′) (SEQ ID NO:31) and 3′W47L (5′-GTT GCT GTT GAT GCT GGC CAC CAG CTC CAG GCC CTT GCC AG-3′) (SEQ ID NO:32) were used. The PCR step was done following the manufacturer's recommendations, by incubating at 95° C. for 30 sec, followed by 18 cycles of 95° C. for 30 sec, 55° C. for 1 min and 68° C. for 1 min, followed by incubating at 68° C. for 7 min. The oligonucleotide primers, each complementary to opposite strands of the vector were extended by PfuTurbo DNA Polymerase (Stratagene) without primer displacement. The resulting PCR reaction generated a mutated plasmid containing staggered nicks, which was treated with DpnI endonuclease specific for methylated and hemimethylated DNA to digest the parental DNA template and to select for mutation-containing synthesized DNA. The nicked vector DNA incorporating the desired mutations was then transformed into E. coli strain TOP10 Chemically Competent Cells (Invitrogen). Sequence verified miniprep DNA was digested with MluI and XbaI, and the resulting restriction fragment containing the mutated Hu8F5VH gene was subcloned into the modified pVg1.D.Tt expression vector described below.

### Modification of Expression Vectors

The allotype of the human gamma-1 constant region gene in the expression plasmid pVg1.D.Tt was modified from G1m (z,a) to the z, non-a allotype. The overlap-extension PCR method (Higuchi, R., in “PCR Technology: Principles and Applications for DNA Amplification”, Stockton Press, New York (1989), pp. 61-70) was used to generate the amino acid substitutions D356E and L358M (numbered according to the EU index of Kabat, E. A., et al., “Sequences of Proteins of Immunological Interest”, 5^{th} ed., National Institutes of Health, Bethesda, Md. (1991)), using the mutagenesis primers 356E358M-A (5′-CCA TCC CGG GAG GAG ATG ACC AAG AAC-3′) (SEQ ID NO:33) and 356E358M-B (5′-GTT CTT GGT CAT CTC CTC CCG GGA TGG-3′) (SEQ ID NO:34). The first round of PCR used outside primer g1-5 (5′-CCA CAT GGA CAG AGG CCG-3′) (SEQ ID NO:35) and 356E358M-B for the left-hand fragment, and outside primer mc-124 (5′-AGG GCA GCG CTG GGT GC-3′) (SEQ ID NO:36) and 356E358M-A for the right-hand fragment. The PCR reactions were done using the Expand High Fidelity PCR System (Roche Diagnostics Corporation, Indianapolis, Ind.) by incubating at 95° C. for 5 min, followed by 35 cycles of 95° C. for 30 sec, 60° C. for 30 sec and 72° C. for 1 min, followed by incubating at 72° C. for 10 min. The second round of PCR to combine the left-hand and right-hand fragments was done as described above, using outside primers g1-5 and mc-124, by incubating at 95° C. for 5 min, followed by 35 cycles of 95° C. for 30 sec, 60° C. for 30 sec and 72° C. for 90 sec, followed by incubating at 72° C. for 7 min. Following digestion with SfiI and EagI, the resulting restriction fragment was subcloned into a modified form of the pVg1.D.Tt expression vector containing an NheI restriction site in the intron between the hinge and CH2 exons.

Mutations to the lower hinge region of the gamma-1 constant region gene were also generated by site-directed mutagenesis, using the plasmid described above as a template. To generate the amino acid substitutions L234A and L235A (numbered according to the EU index of Kabat, E. A., et al., ibid.), the mutagenesis primers 5′L234A L235A (5′-CAT CTC TTC CTC AGC ACC TGA AGC CGC GGG GGG ACC GTC AGT CTT CCT-3′) (SEQ ID NO:37) and 3′L234A L235A (5′-AGG AAG ACT GAC GGT CCC CCC GCG GCT TCA GGT GCT GAG GAA GAG ATG -3′) (SEQ ID NO:38) were used. The PCR step was done using the QuikChange II Site-Directed Mutagenesis Kit (Stratagene), as described above, by incubating at 95° C. for 30 sec, followed by 18 cycles of 95° C. for 30 sec, 55° C. for 1 min and 68° C. for 1 min, followed by incubating at 68° C. for 7 min. Following digestion with DpnI, E. coli strain TOP10 Chemically Competent Cells (Invitrogen) were transformed with a small portion of the PCR product. The plasmid was digested with NheI and EagI, and the resulting restriction fragment was subcloned into the modified pVg1.D.Tt expression vector described above containing an NheI site in the intron between the hinge and CH2 exons. Mutations were verified by nucleotide sequencing.

### Transient Transfection

Human kidney cell line 293T/17 (American Type Culture Collection, Manassus, Va.) was maintained in DMEM (BioWhittaker, Walkersville, Md.) containing 10% Fetal Bovine Serum (FBS) (HyClone, Logan, Utah), 0.1 mM MEM non-essential amino acids (Invitrogen) and 2 mM L-glutamine (Invitrogen), hereinafter referred to as 293 medium, at 37° C. in a 7.5% CO₂ incubator. For expression and purification of monoclonal antibodies after transient transfection, 293T/17 cells were incubated in DMEM containing 2% low-IgG FBS (HyClone), 0.1 mM MEM non-essential amino acids and 2 mM L-glutamine, hereinafter referred to as low-IgG 293 medium.

Transient transfection of 293T/17 cells was carried out using Lipofectamine 2000 (Invitrogen) following the manufacturer's recommendations. Approximately 2×10⁷ cells per transfection were plated in a T-175 flask in 50 ml of 293 medium and grown overnight to confluence. The next day, 35 μg of light chain plasmid and 35 μg of heavy chain plasmid were combined with 3.75 ml of Hybridoma-SFM (HSFM) (Life Technologies, Rockville, Md.). In a separate tube, 175 μl of Lipofectamine 2000 reagent and 3.75 ml of HSFM were combined and incubated for 5 min at room temperature. The 3.75 ml Lipofectamine 2000-HSFM mixture was mixed gently with the 3.75 ml DNA-HSFM mixture and incubated at room temperature for 20 min. The medium covering the 293T/17 cells was aspirated and replaced with low-IgG 293 medium, then the lipofectamine-DNA complexes were added dropwise to the cells, mixed gently by swirling, and the cells were incubated for 7 days at 37° C. in a 7.5% CO₂ incubator before harvesting the supernatants.

### Measurement of Antibody Expression by ELISA

Expression of Hu8F5 antibodies was measured by sandwich ELISA. MaxiSorp ELISA plates (Nunc Nalge International, Rochester, N.Y.) were coated overnight at 4° C. with 100 μl/well of a 1:1000 dilution of AffiniPure goat anti-human IgG Fcγ-chain specific polyclonal antibodies (Jackson ImmunoResearch Laboratories, Inc., West Grove, Pa.) in 0.2 M sodium carbonate-bicarbonate buffer, pH 9.4, washed with Wash Buffer (PBS containing 0.1% Tween 20), and blocked for 1 hr at room temperature with 300 μl/well of SuperBlock Blocking Buffer in TBS (Pierce Chemical Company, Rockford, Ill.). After washing with Wash Buffer, samples containing Hu8F5 were appropriately diluted in ELISA Buffer (PBS containing 1% BSA and 0.1% Tween 20) and 100 μl/well was applied to the ELISA plates. As a standard, humanized IgG1/κ antibody daclizumab (PDL BioPharma, Inc.) was used. After incubating the plates for 1 hr at room temperature, and washing with Wash Buffer, bound antibodies were detected using 100 μl/well of a 1:1000 dilution of HRP-conjugated goat anti-human kappa light chain specific polyclonal antibodies (Southern Biotechnology Associates, Inc., Birmingham, Ala.). After incubating for 1 hr at room temperature, and washing with Wash Buffer, color development was performed by adding 100 μl/well of ABTS Peroxidase Substrate/Peroxidase Solution B (KPL, Inc., Gaithersburg, Md.). After incubating for 7 min at room temperature, color development was stopped by adding 50 μl/well of 2% oxalic acid. Absorbance was read at 415 nm using a VersaMax microplate reader (Molecular Devices Corporation, Sunnyvale, Calif.).

### Purification of 8F5 Antibodies

Culture supernatants from transient transfections were harvested by centrifugation, and sterile filtered. The pH of the filtered supernatants was adjusted by addition of 1/50 volume of 1 M sodium citrate, pH 7.0. Supernatants were run over a 1 ml HiTrap Protein A HP column (GE Healthcare Bio-Sciences Corporation, Piscataway, N.J.) that was pre-equilibrated with 20 mM sodium citrate, 150 mM NaCl, pH 7.0. The column was washed with the same buffer, and bound antibody was eluted with 20 mM sodium citrate, pH 3.0. After neutralization by addition of 1/50 volume of 1.5 M sodium citrate, pH 6.5, the pooled antibody fractions were concentrated to ˜0.5-1.0 mg/ml using a 15 ml Amicon Ultra-15 centrifugal filter device (30,000 dalton MWCO) (Millipore Corporation, Bedford, Mass.). Samples were then filter sterilized using a 0.2 μm Acrodisc syringe filter with HT Tuffryn membrane (Pall Corporation, East Hills, N.Y.). The concentrations of the purified antibodies were determined by UV spectroscopy by measuring the absorbance at 280 nm (1 mg/ml=1.4 A₂₈₀).

### Competition ELISA

MaxiSorp ELISA plates (Nalge Nunc International) were coated overnight at 4° C. with 100 μl/well of 5.0 μg/ml human A-beta oligomer antigen (1-42) (Abbott Biosciences Corporation, Worcester, Mass.) in PBS, washed with Wash Buffer (PBS containing 0.1% Tween 20), and blocked for 1 hr at room temperature with 340 μl/well of SuperBlock Blocking Buffer in PBS (Pierce Chemical Company). After washing with Wash Buffer, a mixture of biotinylated Mu8F5 (1.0 μg/ml final concentration) and competitor antibody (Mu8F5 or Hu8F5 starting at 27 μg/ml final concentration and serially diluted 3-fold) in 100 μl/well of 5% Superblock Blocking Buffer in PBS was added in triplicate. As a no-competitor control, 100 μl/well of 5% Superblock Blocking Buffer in PBS was used. After incubating the plates for 2 hrs at room temperature, and washing with Wash Buffer, bound antibodies were detected using 100 μl/well of 1 μg/ml HRP-conjugated streptavidin (Pierce Chemical Company) in 5% Superblock Blocking Buffer in PBS. After incubating for 30 min at room temperature, and washing with Wash Buffer, color development was performed by adding 100 μl/well of ABTS Peroxidase Substrate/Peroxidase Solution B (KPL). After incubating for 5 min at room temperature, color development was stopped by adding 50 μl/well of 2% oxalic acid. Absorbance was read at 415 nm.

### Results:

### Humanization

For humanization of the Mu8F5 variable regions, the general approach provided in the present invention was followed. First, a molecular model of the Mu8F5 variable regions was constructed with the aid of the computer programs ABMOD and ENCAD (Levitt, M., J. Mol. Biol. 168: 595-620 (1983)). Next, based on a homology search against human V and J segment sequences, the VH segment YSE′CL (Mariette, X., et al., Eur. J. Immunol. 23: 846-851 (1993)) and the J segment JH4 (Ravetch, J. V., et al., Cell 27: 583-591 (1981)) were selected to provide the frameworks for the Hu8F5 heavy chain variable region. For the Hu8F5 light chain variable region, the VL segment TR1.37′CL (Portolano, S., et al., J. Immunol. 151: 2839-2851 (1993)) and the J segment JK4 (Hieter, P. A., et al., J. Biol. Chem. 257: 1516-1522 (1982)) were used. The identity of the framework amino acids between Mu8F5 VH and the acceptor human YSE′CL and JH4 segments was 80%, while the identity between Mu8F5 VL and the acceptor human TR1.37′CL and JK4 segments was 86%.

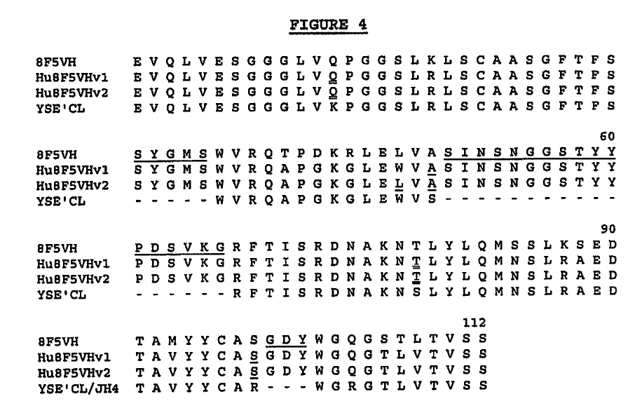

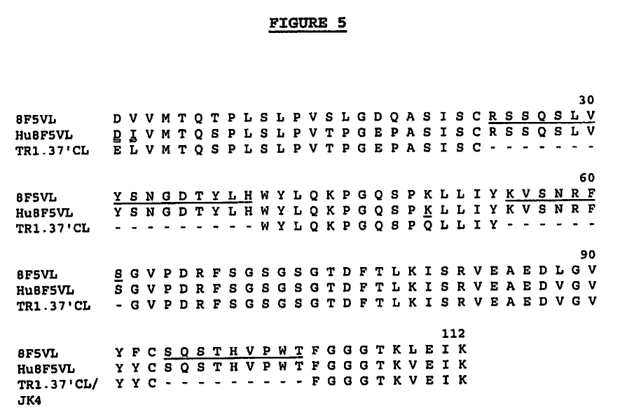

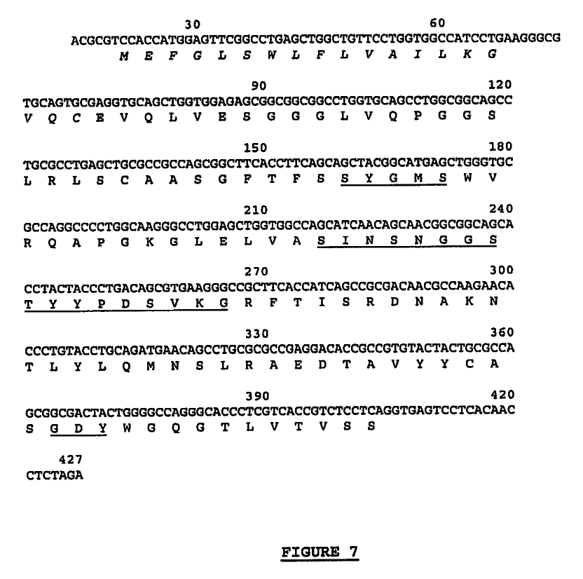

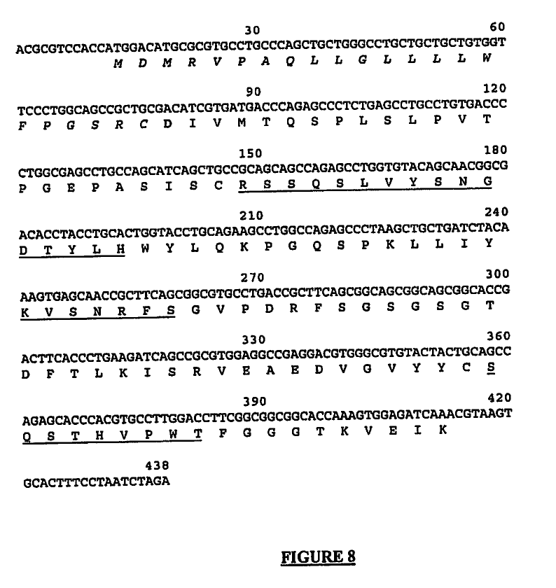

At framework positions in which the computer model suggested significant contact with the CDRs, the amino acids from the mouse V regions were substituted for the original human framework amino acids. This was done at residues 49 and 98 for versions 1 and 2 of the heavy chain, and additionally at residue 47 for version 2 of the heavy chain (FIG. 7). For the light chain, replacement was made at residue 50 (FIG. 8). Framework residues that occurred only rarely at their respective positions in the corresponding human V region subgroups were replaced with human consensus amino acids at those positions. This was done at residues 13 and 78 of the heavy chain (FIG. 4), and at residues 1 and 2 of the light chain (FIG. 5).

### Expression of the Hu8F5 Antibodies

Genes encoding humanized VH or VL were designed as mini-exons including signal peptides, splice donor signals, and appropriate restriction enzyme sites for subsequent cloning into a mammalian expression vector. The splice donor signals in the VH and VL mini-exons were derived from the corresponding human germline JH and JK sequences, respectively. The signal peptide sequences in the humanized VH and VL mini-exons were provided by Abbott Bioresearch Center. The Hu8F5 VH and VL genes were constructed by assembly of overlapping synthetic oligonucleotides and PCR. Primers 1-28 for the synthesis of the humanized heavy chain variable region are presented in Table A. Primers 1-29 for the synthesis of the humanized light chain variable region are presented in Table B.

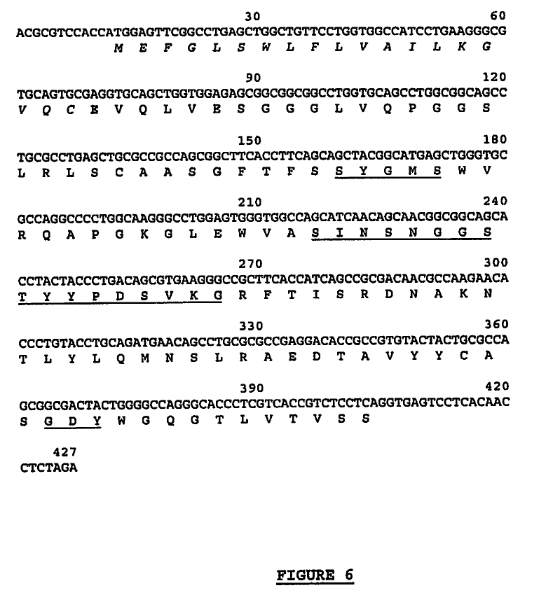

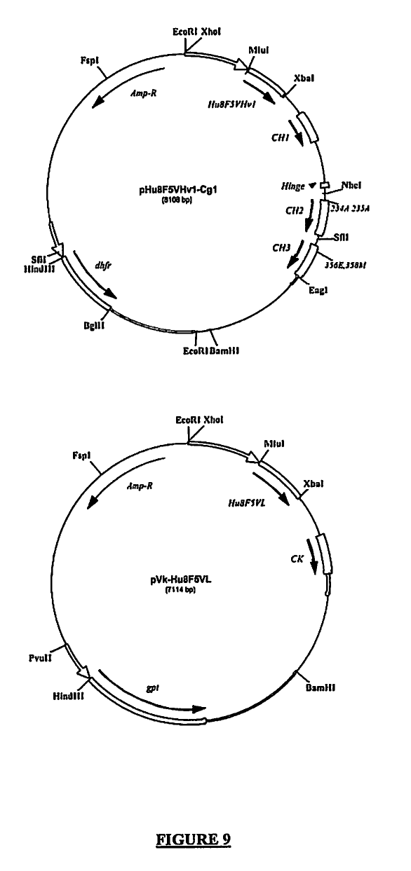

The DNA sequences and deduced amino acid sequences of the humanized VHv1, VHv2 and VL mini-exons are shown in FIGS. 6, 7 and 8, respectively. The resulting V gene fragments were cloned, respectively, into a modified form of pVg1.D.Tt and pVk (FIG. 9).

Transient transfectants producing Hu8F5 were generated as described in Materials and Methods. Culture supernatants of transiently transfected 293T/17 cells were analyzed by ELISA for production of Hu8F5. Expression levels of approximately 30-50 μg/ml were typically observed. Hu8F5 IgG1/κ monoclonal antibodies were purified from exhausted culture supernatant with a protein A Sepharose column as described in Materials and Methods. SDS-PAGE analysis under non-reducing conditions indicated that the Hu8F5 antibodies had a molecular weight of about 150-160 kDa. Analysis under reducing conditions indicated that the Hu8F5 antibodies were comprised of a heavy chain with a molecular weight of about 50 kDa and a light chain with a molecular weight of about 25 kDa. The purity of the antibodies appeared to be more than 95%.

### Binding Properties of Hu8F5 Antibodies

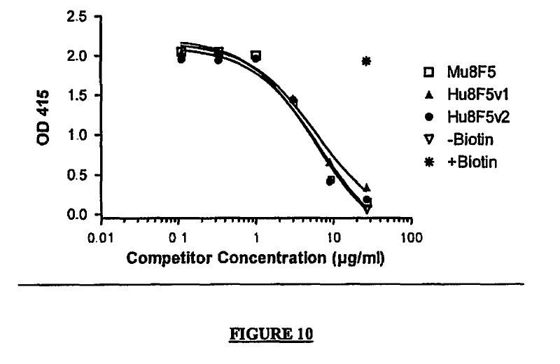

The affinity of Hu8F5 to human A-beta oligomer antigen (1-42) was analyzed by competition ELISA as described in Materials and Methods. A representative result is shown in FIG. 10. Both Mu8F5 and Hu8F5 competed with biotinylated Mu8F5 in a concentration-dependent manner. As shown in Table C, the mean IC₅₀ values of Mu8F5, Hu8F5v1 and Hu8F5v2, obtained using the computer software GraphPad Prism (GraphPad Software Inc., San Diego, Calif.), were 5.08, 6.36 and 6.99 μg/ml, respectively. The binding of Hu8F5v1 and Hu8F5v2 to human A-beta oligomer antigen (1-42) was equivalent to that of Mu8F5. These results clearly indicate that humanization of mouse anti-A-beta monoclonal antibody 8F5 was successful: Hu8F5 retained full binding affinity to human A-beta oligomer antigen (1-42).

TABLE A

| | |
|---|---|
| Oligo # | Sequences |

| | |
|---|---|
| Hu8F5VHv1 | agacgctgttgcctTATACGCGTCCACCATGGAGTTCG |
| #1 | (SEQ ID NO:39) |
| Hu8F5VHv1 | ACAGCCAGCTCAGGCCGAACTCCATGGTGGACG |
| #2 | (SEQ ID NO:40) |
| Hu8F5VHv1 | GCCTGAGCTGGCTGTTCCTGGTGGCCATCC |
| #3 | (SEQ ID NO:41) |
| Hu8F5VHv1 | ACTGCACGCCCTTCAGGATGGCCACCAGGA |
| #4 | (SEQ ID NO:42) |
| Hu8F5VHv1 | TGAAGGGCGTGCAGTGCGAGGTGCAGCTGG |
| #5 | (SEQ ID NO:43) |
| Hu8F5VHv1 | GCCGCCGCTCTCCACCAGCTGCACCTCGC |
| #6 | (SEQ ID NO:44) |
| Hu8F5VHv1 | TGGAGAGCGGCGGCGGCCTGGTGCAGCC |
| #7 | (SEQ ID NO:45) |
| Hu8F5VHv1 | CAGGCTGCCGCCAGGCTGCACCAGGCC |
| #8 | (SEQ ID NO:46) |
| Hu8F5VHv1 | TGGCGGCAGCCTGCGCCTGAGCTGCGC |
| #9 | (SEQ ID NO:47) |
| Hu8F5VHv1 | TGAAGCCGCTGGCGGCGCAGCTCAGGCG |
| #10 | (SEQ ID NO:48) |
| Hu8F5VHv1 | CGCCAGCGGCTTCACCTTCAGCAGCTACGGC |
| #11 | (SEQ ID NO:49) |
| Hu8F5VHv1 | GCGCACCCAGCTCATGCCGTAGCTGCTGAAGG |
| #12 | (SEQ ID NO:50) |
| Hu8F5VHv1 | ATGAGCTGGGTGCGCCAGGCCCCTGGCA |
| #13 | (SEQ ID NO:51) |
| Hu8F5VHv1 | CCCACTCCAGGCCCTTGCCAGGGGCCTG |
| #14 | (SEQ ID NO:52) |
| Hu8F5VHv1 | AGGGCCTGGAGTGGGTGGCCAGCATCAACAGC |
| #15 | (SEQ ID NO:53) |
| Hu8F5VHv1 | TGCTGCCGCCGTTGCTGTTGATGCTGGCCA |
| #16 | (SEQ ID NO:54) |
| Hu8F5VHv1 | AACGGCGGCAGCACCTACTACCCTGACAGCG |
| #17 | (SEQ ID NO:55) |
| Hu8F5VHv1 | TGAAGCGGCCCTTCACGCTGTCAGGGTAGTAGG |
| #18 | (SEQ ID NO:56) |
| Hu8F5VHv1 | TGAAGGGCCGCTTCACCATCAGCCGCGACA |
| #19 | (SEQ ID NO:57) |
| Hu8F5VHv1 | CAGGGTGTTCTTGGCGTTGTCGCGGCTGATGG |
| #20 | (SEQ ID NO:58) |
| Hu8F5VHv1 | ACGCCAAGAACACCCTGTACCTGCAGATGAACAGCCT |
| #21 | (SEQ ID NO:59) |
| Hu8F5VHv1 | TGTCCTCGGCGCGCAGGCTGTTCATCTGCAGGTA |
| #22 | (SEQ ID NO:60) |
| Hu8F5VHv1 | GCGCGCCGAGGACACCGCCGTGTACTACTGCG |
| #23 | (SEQ ID NO:61) |
| Hu8F5VHv1 | AGTAGTCGCCGCTGGCGCAGTAGTACACGGCGG |
| #24 | (SEQ ID NO:62) |
| Hu8F5VHv1 | CCAGCGGCGACTACTGGGGCCAGGGCACC |
| #25 | (SEQ ID NO:63) |
| Hu8F5VHv1 | TGAGGAGACGGTGACGAGGGTGCCCTGGCCCC |
| #26 | (SEQ ID NO:64) |
| Hu8F5VHv1 | CTCGTCACCGTCTCCTCAGGTGAGTCCTCACAACCTC |
| #27 | (SEQ ID NO:65) |
| Hu8F5VHv1 | gcgtcacggggtaaATATCTAGAGGTTGTGAGGACTCACC |
| #28 | (SEQ ID NO:66) |
| 5′ | agacgctgttgcctTATACG |
| Hu8F5VHv1 | (SEQ ID NO:67) |
| 3′ | gcgtcacggggtaaATATCTA |
| Hu8F5VHv1 | (SEQ ID NO:68) |

TABLE B

| | |
|---|---|
| Oligo # | Sequences |

| | |
|---|---|
| Hu8F5VL | GCGTATAtcccggttgttgct |
| #1 | (SEQ ID NO:69) |
| Hu8F5VL | agcaacaaccgggaTATACGCGTCCACCATGGACATGCG |
| #2 | (SEQ ID NO:70) |
| Hu8F5VL | GCTGGGCAGGCACGCGCATGTCCATGGTGGAC |
| #3 | (SEQ ID NO:71) |
| Hu8F5VL | CGTGCCTGCCCAGCTGCTGGGCCTGCTG |
| #4 | (SEQ ID NO:72) |
| Hu8F5VL | CCAGGGAACCACAGCAGCAGCAGGCCCAGCA |
| #5 | (SEQ ID NO:73) |
| Hu8F5VL | CTGCTGTGGTTCCCTGGCAGCCGCTGCGACA |
| #6 | (SEQ ID NO:74) |
| Hu8F5VL | GCTCTGGGTCATCACGATGTCGCAGCGGCTG |
| #7 | (SEQ ID NO:75) |
| Hu8F5VL | TCGTGATGACCCAGAGCCCTCTGAGCCTGCCTG |
| #8 | (SEQ ID NO:76) |
| Hu8F5VL | GCTCGCCAGGGGTCACAGGCAGGCTCAGAGG |
| #9 | (SEQ ID NO:77) |
| Hu8F5VL | TGACCCCTGGCGAGCCTGCCAGCATCAGCTGC |
| #10 | (SEQ ID NO:78) |
| Hu8F5VL | GCTCTGGCTGCTGCGGCAGCTGATGCTGGCAG |
| #11 | (SEQ ID NO:79) |
| Hu8F5VL | CGCAGCAGCCAGAGCCTGGTGTACAGCAACGGC |
| #12 | (SEQ ID NO:80) |
| Hu8F5VL | CCAGTGCAGGTAGGTGTCGCCGTTGCTGTACACCAG |
| #13 | (SEQ ID NO:81) |
| Hu8F5VL | GACACCTACCTGCACTGGTACCTGCAGAAGCCTGG |
| #14 | (SEQ ID NO:82) |
| Hu8F5VL | GCAGCTTAGGGCTCTGGCCAGGCTTCTGCAGGTA |
| #15 | (SEQ ID NO:83) |
| Hu8F5VL | CCAGAGCCCTAAGCTGCTGATCTACAAAGTGAGCAACCG |
| #16 | (SEQ ID NO:84) |
| Hu8F5VL | GGCACGCCGCTGAAGCGGTTGCTCACTTTGTAGATCA |
| #17 | (SEQ ID NO:85) |
| Hu8F5VL | CTTCAGCGGCGTGCCTGACCGCTTCAGCGG |
| #18 | (SEQ ID NO:86) |
| Hu8F5VL | TGCCGCTGCCGCTGCCGCTGAAGCGGTCA |
| #19 | (SEQ ID NO:87) |
| Hu8F5VL | CAGCGGCAGCGGCACCGACTTCACCCTGAAGA |
| #20 | (SEQ ID NO:88) |
| Hu8F5VL | CTCCACGCGGCTGATCTTCAGGGTGAAGTCGG |
| #21 | (SEQ ID NO:89) |
| Hu8F5VL | TCAGCCGCGTGGAGGCCGAGGACGTGGG |
| #22 | (SEQ ID NO:90) |
| Hu8F5VL | TGGCTGCAGTAGTACACGCCCACGTCCTCGGC |
| #23 | (SEQ ID NO:91) |
| Hu8F5VL | CGTGTACTACTGCAGCCAGAGCACCCACGTGCC |
| #24 | (SEQ ID NO:92) |
| Hu8F5VL | CCGCCGAAGGTCCAAGGCACGTGGGTGCTC |
| #25 | (SEQ ID NO:93) |
| Hu8F5VL | TTGGACCTTCGGCGGCGGCACCAAAGTGGAGA |
| #26 | (SEQ ID NO:94) |
| Hu8F5VL | AGGAAAGTGCACTTACGTTTGATCTCCACTTTGGTGCCG |
| #27 | (SEQ ID NO:95) |
| Hu8F5VL | TCAAACGTAAGTGCACTTTCCTAATCTAGATATtcggctcgacg |
| #28 | (SEQ ID NO:96) |
| Hu8F5VL | cgtcgagccgaATATCTAGATT |
| #29 | (SEQ ID NO:97) |
| 5′ Hu8F5VL | agcaacaaccgggaTATACGC |
| | (SEQ ID NO:98) |
| 3′ Hu8F5VL | cgtcgagccgaATATCTAGATT |
| | (SEQ ID NO:99) |

TABLE C

| Antibody | Expt. 1 | Expt. 2 | Expt. 3 | Average | S.D. |
|---|---|---|---|---|---|
| Mu8F5 | 4.58 | 4.87 | 5.81 | 5.08 | 0.64 |
| Hu8F5v1 | 6.28 | 6.85 | 5.93 | 6.36 | 0.47 |
| Hu8F5v2 | 6.29 | 8.12 | 6.56 | 6.99 | 0.99 |

### Assembly of Humanized Antibody VH and VL Fragments

VH and VL gene fragments for humanization designs were assembled by annealing overlapping oligonucleotides covering the entire sequence. Briefly, the entire coding strand of the VH or VL fragment was divided into a series of sixty-nucleotide oligos., each designed to have a thirty nucleotide overlap with two corresponding bottom strand oligos. The sum of the bottom strand oligos. also covered the entire sequence. Taken together, the oligonucleotides filled the complete double-stranded DNA segment.

In the first step of the procedure, the oligonucleotides were kinased (New England Biolabs cat #201S) by combining seven top strand and seven bottom strand oligos together at a concentration of 3 nM each in a 100 microliter reaction for 30 minutes at 37° C. The kinased oligos were then phenol/chloroform extracted, precipitated, and resuspended in 100 microliters of NEB Ligase Buffer.

In the second step of the procedure, the oligonucleotides were annealed by heating to 95° C., then slowly cooled to 20° C. over a period of 90 minutes by a controlled cooling ramp in a PCR machine.

In the third step of the procedure, 1 microliter of Ligase (NEB cat#202S) was added to the annealed oligos in order to ligate them together to form the strands of the VH and VL segments. Ligase was inactivated by heating to 65° C. for 10 minutes.

In the fourth step, the ends of the assembled fragments were filled in with Klenow enzyme (NEB cat#212S), and the DNA was gel purified before cloning into the human heavy and light chain cassette vectors.

### Example II

### Competition ELISA

The following protocol was utilized to carry out the Competition ELISA assay:

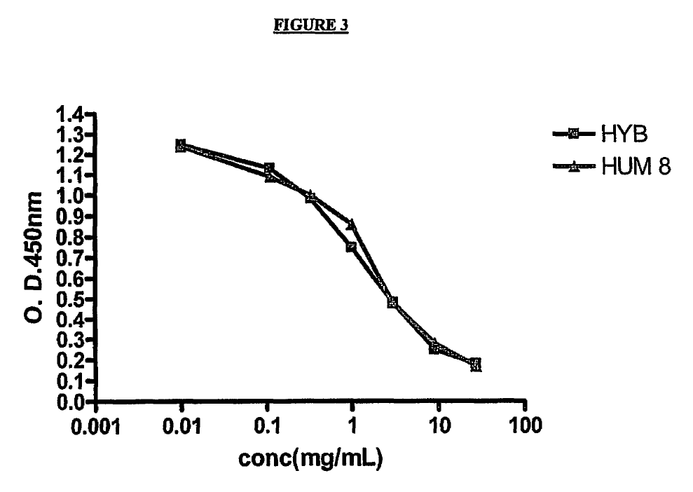

Initially, plates (1 plate/experiment) were coated overnight with A-Beta antigen (1-42) at a concentration of 5 μg/mL in phosphate buffered saline (PBS). The following day, the supernatant was discarded, and the plates were blocked with 340 mL of Super Block buffer (Pierce, Rockford, Ill.) for 45 min. The plates were then emptied, and the biotinylated 7C6 or 5F7 mouse antibody was added at a concentration of 1 μg/mL. (Volume=100 μL) Other antibodies (mouse or humanized) were added at concentrations ranging from 27 μg/mL to 0.11 μg/mL. (Volume=50 μL) The plates were then incubated for two hours and washed 5× times with Phosphate Buffered Saline (PBS). Neutra Avidin HRP was added as a secondary reagent (dilution 1:20,000; volume=100 μL). The plates were then incubated for 30 min. and washed 5× times. TMB substrate (Invitrogen, Carlsbad, Calif.) was then added (volume=100 μL). Subsequently, the plates were incubated for 4 min. The reaction was then stopped with 2N sulfuric acid. (Vol-100 μL) Plates were read spectrophotometrically at a wavelength of 450 nm. The results are shown in FIG. 3.

In particular, FIG. 3 illustrates the equivalence of the humanized antibody (i.e., 8F5hum8, HUM8) to the mouse parent with respect to the humanized antibody's ability to compete with (and inhibit the binding signal of) the biotinylated mouse antibody. Thus, the humanized antibody has retained its binding potency.

### Example III

### Binding of 8F5hum8 to Aβ(1-42) Fibrils

Since 8F5hum8 antibody was generated against soluble globulomers, it was hypothesized that 8F5hum8 should not bind to deposited plaque or fibril material. Therefore, binding of 8F5hum8 to polymerized Aβ fibril suspensions was tested as described in the following example:

Preparation of Aβ(1-42) Fibrils:

1 mg Aβ(1-42) (Bachem Inc., Catalog Nr.: H-1368) was dissolved in 500 μl aqueous 0.1% NH₄OH (Eppendorf tube), and the sample was stirred for 1 min at room temperature followed by 5 min centrifugation at 10000 g. Supernatant was pipetted into a new Eppendorf tube and the Aβ(1-42) concentration measured according to Bradford protein concentration assay (BIO-RAD Inc. assay procedure).

100 μl of this freshly prepared Aβ(1-42) solution were neutralized with 300 μl 20 mM NaH2PO4; 140 mM NaCl; pH 7.4 followed by 2% HCl to adjust pH 7.4. The sample was incubated for another 20 hrs at 37° C. and centrifuged (10 min, 10000 g). The supernatant was discarded and the fibril pellet resuspended with 400 μl 20 mM NaH2PO4; 140 mM NaCl; pH 7.4 under 1 min stirring on a Vortex mixer followed by centrifugation (10 min, 10000 g). After discarding the supernatant, this resuspending procedure was repeated, and the final fibril suspension spun down by another centrifugation (10 min, 10000 g). The supernatant was once again discarded and the final pellet resuspended in 380 μl 20 mM NaH2PO4; 140 mM NaCl; pH7.4 under 1 min stirring on a Vortex mixer. Aliquots of the sample were stored at −20° C. in a freezer.

80 μl fibril suspension were mixed with 320 μl 20 mM NaH2PO4; 140 mM NaCl; 0.05% Tween 20; pH 7.4, buffer and stirred for min at room temperature followed by sonification (20 sec). After centrifugation (10 min, 10000 g), the pellet was resuspended with 190 μl 20 mM NaH2PO4; 140 mM NaCl; 0.05% Tween 20; pH 7.4 under stirring in a Vortex mixer.

Binding of Antibodies to Aβ(1-42) Fibrils

10 μl aliquots of this fibril suspension was incubated with:
a) 10 μl 20 mM Na Pi; 140 mM NaCl; pH 7.4
b) 10 μl 0.1 μg/μl mMAb 6E10 Signet Inc. Cat.#9320 in 20 mM NaH2PO4; 140 mM NaCl; pH 7.4
c) 10 μl 0.1 μg/μl mMAb 8F5hum8 in 20 mM Na Pi; 140 mM NaCl; pH 7.4
d) 10 μl 0.1 μg/μl mMAb IgG2a in 20 mM Na Pi; 140 mM NaCl; pH 7.4

Samples were incubated for 20 h at 37° C. Finally the samples were centrifuged (10 min at 10000 g). The supernatants containing the unbound antibody fraction were collected and mixed with 20 μl SDS-PAGE sample buffer. The pellet fractions were washed with 50 μl 20 mM NaH2PO4; 140 mM NaCl; pH 7.4 buffer under 1 min stirring in a Vortex mixer followed by centrifugation (10 min, 10000 g). The final pellets were resuspended in 20 μl 20 mM Na Pi; 140 mM NaCl; 0.025% Tween 20; pH 7.4 buffer and solved in 20 μl SDS-PAGE buffer.

SDS-PAGE Analysis

Supernatants and resuspended pellet samples were heated for 5 min at 98° C. and loaded onto a 18% Tris/Glycin Gel under the following conditions:

SDS-sample buffer: 0.3 g SDS; 0.77 g DTT; 4 ml 1M Tris/HCl pH 6.8; 8 ml glycerol; 1 ml 1% Bromphenolblue in Ethanol; add water to 50 ml 18% Tris/Glycin Gel:Invitrogen Inc., No.: EC6505BOX
running buffer: 7.5 g Tris; 36 g Glycine; 2.5 g SDS; add water to 2.5 l

The PAGE was run at 20 mA. Gels were stained by Coomassie Blue R250.

### Results:

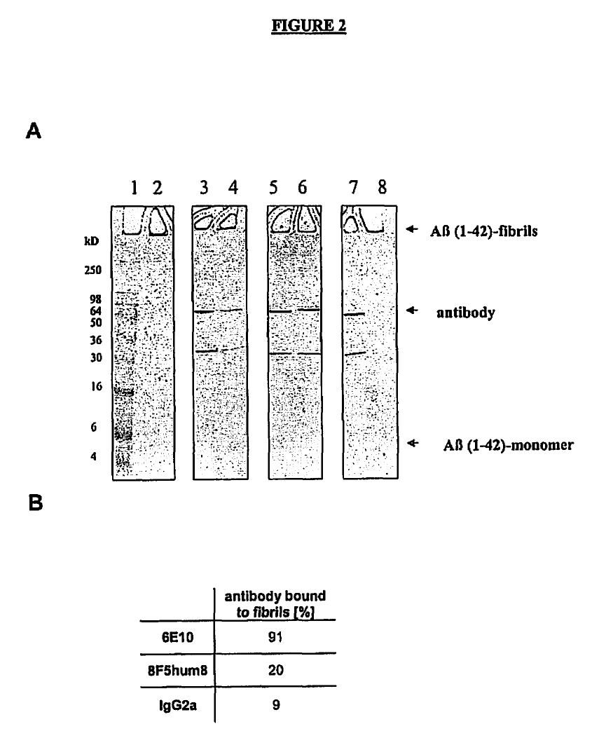

Coomassie staining of SDS-PAGE indicated the presence of heavy and light chains of antibodies predominantly in the supernatant of the fibril suspension for the antibody 8F5hum8 (lane 3, FIG. 2a), the remaining fibril suspension of the pellet showed very little antibody material while also showing partly depolymerized Abeta at 4.5 kDa. In contrast to 8F5hum8, other anti-Aβ antibodies did not show up in the soluble fraction (6E10, lane 5, FIG. 2a) compared to fibril bound fraction (lane 6, FIG. 2a). As a reference for unspecific binding and the intrinsic background of this method, the unspecific antibody IgG2a was used as an internal control. The IgG2a antibody, which is not directed against the Aβ peptide in any form, shows a certain unspecific binding to Aβ fibrils.

The relative binding to fibril type Abeta was evaluated from SDS-PAGE analysis by measuring the Reflective Density values from the heavy chain of the antibodies in the fibril bound and the supernatant fractions and calculated according to the following formula:

Fibril bound Ab fraction=RD_{fibril faction}×100%/(RD_{fibril faction}+RD_{supernatant fraction}).

The following values were obtained:

| | Fibril bound Ab |
|---|---|
| Antibody | fraction |
| 6E10 | 91% |
| 8F5hum8 | 20% |
| IgG2a | 9% |

These data indicate a significant reduction of bound 8F5hum8 compared to standard antibody 6E10.

### Example IV

### 8F5hum8 Preferential Globulomer Binding Compared to Monomer Preparations of Aβ(1-40) and Aβ(1-42) Determined by Dot Blot

To test the selectivity of 8F5hum8, Aβ(1-42) monomer as well as freshly prepared Aβ(1-40) were used as surrogates for monomers. The oligomer selectivity versus Aβ(1-42) monomer and Aβ(1-40) monomer was examined by dot blot immunoassay. In this experiment, 8F5hum8 exhibited preferential binding to Aβ(1-42) globulomer (compared to a known antibody 4G8 mapping to a similar region as 8F5hum8, but derived from immunization with a linear peptide Aβ(17-24) (Abcam Ltd., Cambridge, Mass.)), as compared to Aβ(1-42) monomer as well as compared to Aβ(1-40) monomer.

### Description of Dot Blot Method

### 1) Preparation of Aβ Antigens:

### a) Preparation of Aβ(1-42) Globulomer:

9 mg Aβ(1-42) Fa. Bachem were dissolved in 1.5 ml HFIP (1.1.1.3.3.3 Hexafluor-2-propanol) and incubated 1.5 h at 37° C. The solution was evaporated in a SpeedVac and suspended in 396 μl DMSO (5 mM Aβ stock solution). The sample was sonified for 20 seconds in a sonic water bath, shaken for 10 minutes and stored over night at −20° C.

The sample was diluted with 4.5 ml PBS (20 mM NaH2PO4; 140 mM NaCl; pH 7.4) and 0.5 ml 2% aqueous SDS-solution were added (0.2% SDS content). The mixture was incubated for 7 h at 37° C., diluted with 16 ml H₂O and further incubated for 16 hours at 37 deg C. After that, the Aβ(1-42) globulomer solution was centrifuged for 20 min at 3000 g. The supernatant was concentrated to 0.5 ml by 30 KDa centriprep. The concentrate was dialysed against 5 mM NaH2PO4; 35 mM NaCl; pH7.4 overnight at 6° C. Subsequently, the Aβ(1-42) globulomer concentrate was centrifuged for 10 min at 10000 g. The supernatant was then aliquoted and stored at −20° C.

### b) Preparation of Monomer Aβ(1-42), HFIP Pretreated:

3 mg human Aβ(1-42), (Bachem Inc) cat. no. H-1368 were dissolved in 0.5 ml HFIP (6 mg/ml suspension) in an 1.7 ml Eppendorff tube and was shaken (Eppendorff Thermo mixer, 1400 rpm) for 1.5 h at 37° C. till a clear solution was obtained. The sample was dried in a speed vac concentrator (1.5 h) and resuspended in 13.2 μl DMSO, shook for 10 sec., followed by ultrasound bath sonification (20 sec) and shaking (e.g. in Eppendorff Thermo mixer, 1400 rpm) for 10 min.

6 ml 20 mM NaH2PO4; 140 mM NaCl; 0.1% Pluronic F68; pH 7.4 was added and stirred for 1 h at room temperature. The sample was centrifuged for 20 min at 3000 g. The supernatant was discarded and the precipitate solved in 0.6 ml 20 mM NaH2PO4; 140 mM NaCl; 1% Pluronic F68; pH 7.4. 3.4 ml water was added and stirred for 1 h at room temperature followed by 20 min centrifugation at 3000 g. 8×0.5 ml aliquots of the supernatant were stored at −20°.

### c) Preparation of Monomer Aβ(1-40):

1 mg human Aβ(1-40), (Bachem Inc) cat. no. H-1194 was suspended in 0.25 ml HFIP (4 mg/ml suspension) in an Eppendorff tube. The tube was shaken (e.g., in an Eppendorff Thermo mixer, 1400 rpm) for 1.5 h at 37° C. to get a clear solution and afterwards dried in a speed vac concentrator (1.5 h). The sample was redissolved in 46 μl DMSO (21.7 mg/ml solution), shaken for 10 sec., followed by 20 sec. sonification in ultrasound bath. After 10 min of shaking (e.g. in Eppendorff Thermo mixer, 1400 rpm), the sample was stored at −20° C. for further use.

### 2) Dot Blot: Materials and Procedure:

### Materials for Dot Blot:

### Aβ-Standards:

Serial dilution of Aβ antigens in 20 mM NaH2PO4, 140 mM NaCl, pH 7.4+0.2 mg/ml BSA
   1) 100 pmol/μl
   2) 10 pmol/μl
   3) 1 pmol/μl
   4) 0.1 pmol/μl
   5) 0.01 pmol/μl

### Nitrocellulose:

Trans-Blot Transfer medium, Pure Nitrocellulose Membrane (0.45 μm); BIO-RAD

### Anti-Mouse-Aβ:

Cat no: Aβ326A (Chemicon)

### Anti-Human-Aβ:

Cat no: A3313 (Sigma)

### Detection Reagent:

NBT/BCIP; Cat no: 11697471001; Roche)

### Bovine Serum Albumin, (BSA):

Cat no: A-7888 (SIGMA)

### Blocking Reagent:

5% low fat milk in TBS

### Buffer Solutions:

TBS
25 mM Tris/HCl buffer pH 7.5
+150 mM NaCl
TTBS
25 mM Tris/HCl−buffer pH 7.5
+150 mM NaCl
+0.05% Tween 20
PBS+0.2 mg/ml BSA
20 mM NaH2PO4 buffer pH 7.4
+140 mM NaCl
+0.2 mg/ml BSA

### Antibody Solution I:

0.2 μg/ml antibody diluted in 20 ml 1% low fat milk in TBS

### Antibody Solution II:

1:5000 dilution
Anti-Mouse-APin 1% low fat milk in TBS for mouse antibody 4G8 or anti-human-Aβ in 1% low fat milk in TBS for humanized anti Aβ globulomer antibody 8F5hum8

### Dot Blot Procedure:

1) 1 μl each of the different Aβ-standards (in their 5 serial dilutions) were dotted onto the nitrocellulose membrane in a distance of approximately 1 cm from each other.
2) The Aβ-standards dots were allowed to dry on the nitrocellulose membrane on air for at least 10 min at room temperature (RT) (=dot blot)
3) Blocking:
   The dot blot was incubated with 30 ml 5% low fat milk in TBS for 16 h at RT.
4) Washing:
   The blocking solution was discarded and the dot blot was incubated under shaking with 20 ml TTBS for 10 min at RT.
5) Antibody solution I:
   The washing buffer was discarded and the dot blot was incubated with antibody solution I for 2 h at RT
6) Washing:
   The antibody solution I was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
7) Antibody solution II:
   The washing buffer was discarded and the dot blot was incubated with antibody solution II 1 h at RT
8) Washing:
   The antibody solution II was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
9) Development:
   The washing solution was discarded. The dot blot was developed with a development solution made from 1 tablet of NBT/BCIP (Roche) dissolved in 20 mL H₂O for 5 min. The development was stopped by intense washing of the dot blot with H₂O. Quantitative evaluation was done using a densitometric analysis (GS800 densitometer (BioRad) and software package Quantity one, Version 4.5.0 (BioRad)) of the dot-intensity. Only the dots for 10 pmol Aβ antigen were evaluated.
      Results for the Discrimination of Aβ Monomer Against Aβ Globulomer by Dot Blot Method: Comparison of 8F5hum8 Versus 4G8.

Serial dilutions of Aβ(1-42) globulomer, Aβ1-42 monomer and Aβ1-40 monomer were made in the range from 100 pmol/μl-0.0 pmol/μl in PBS. Of each sample, 1 μl was dotted onto a nitrocellulose membrane. The mouse monoclonal antibody 4G8 (0.2 μg/ml) were used for detection with an anti-mouse IgG coupled to alkaline phosphatase as secondary antibody and the staining reagent NBT/BCIP (Roche Diagnostics, Mannheim).

The humanized monoclonal antibody 8F5hum8 (0.2 μg/ml) were used for detection with an anti-human IgG coupled to alkaline phosphatase as secondary antibody and the staining reagent NBT/BCIP (Roche Diagnostics, Mannheim). The detection signal was analyzed in its intensity (reflective density=RD) via a densitometer (GS 800, Biorad, Hercules, Calif., USA) at an antigen concentration of 10 pmol. At this concentration for every Aβ-form, the measured reflective density was in the linear range of the densitometer detection. The results are shown in the table below:

| | | | | Ratio | Ratio |
|---|---|---|---|---|---|
| | | | | RD | RD |
| | Reflective Density | | | Aβ (1-42) | Aβ (1-42) |
| | (RD) | | Aβ | globulomer/ | globulomer/ |
| | [10 pmol] | | (1-40) | RD | RD |
| | Aβ (1-42) | Aβ (1-42) | mono- | Aβ (1-42) | Aβ (1-40) |
| | globulomer | monomer | mer | monomer | monomer |
| 8F5 | 2.2 | 1.3 | 0.04 | 1.6 | 59.0 |
| hum8 | | | | | |
| 4G8 | 1.5 | 3.5 | 0.15 | 0.42 | 10.0 |

Discrimination of anti-Aβ-antibodies of Aβ1-40 monomer and Aβ1-42 monomer. The discrimination was calculated as the ratio of detection signal of Aβ1-42 globulomer and Aβ1-42 monomer, respectively Aβ1-40 monomer.

In particular, the above results indicate that 8F5hum8 shows a different binding profile compared to commercially available anti-Aβ(1-42) antibody to 4G8, which maps to Aβ(17-24)(i.e., a linear sequence). More specifically, 8F5hum8 show a preference for globulomer binding versus Aβ42 monomer (see column 4 in table with a ratio for Aβ(1-42) globulomer/Aβ(1-42) monomer of 1.6 for 8F5hum8 versus a ratio of 0.42 for 4G8) as well as a preference for globulomer binding versus Aβ40 (see column 5 in table with a ratio for Aβ(1-42) globulomer/Aβ(1-40) monomer of 59.0 for 8F5hum8 versus a ratio of 10.0 for 4G8). These two improved binding selectivities over standard 4G8 should result in the production of fewer side effects upon use of 8F5hum8, as described above (e.g., plaque binding).

What is claimed is:

1. A binding protein comprising: a) an antigen binding domain which binds to amyloid-beta (1-42) globulomer, said antigen binding domain comprising at least one CDR comprising an amino acid sequence selected from the group consisting of:
CDR-H1. X₁-X₂-X₃-X₄-X₅ (SEQ ID NO:5), wherein;
X₁ is S;
X₂ is Y;
X₃ is G;
X₄ is M; and
X₅ is S.
CDR-H2. X₁-X₂-X₃-X₄-X₅-X₆-X₇-X₈-X₉-X₁₀-X₁₁-X₁₂-X₁₃-X₁₄-X₁₅-X₁₆-X₁₇ (SEQ ID NO:6), wherein;
X₁ is S;
X₂ is I;
X₃ is N;
X₄ is S;
X₅ is N;
X₆ is G;
X₇ is G;
X₈ is S;
X₉ is T;
X₁₀ is Y;
X₁₁ is Y;
X₁₂ is P;
X₁₃ is D;
X₁₄ is S;
X₁₅ is V;
X₁₆ is K; and
X₁₇ is G.
CDR-H3. X₁-X₂-X₃-X₄ (SEQ ID NO:7), wherein;
X₁ is S;
X₂ is G;
X₃ is D; and
X₄ is Y.
CDR-L1. X₁-X₂-X₃-X₄-X₅-X₆-X₇-X₈-X₉-X₁₀-X₁₁-X₁₂-X₁₃-X₁₄-X₁₅-X₁₆ (SEQ ID NO:8), wherein:
X₁ is R;
X₂ is S;
X₃ is S;
X₄ is Q;
X₅ is S;
X₆ is L;
X₇ is V;
X₈ is S;
X₁₀ is N;
X₁₁ is G;
X₁₂ is D;
X₁₃ is T;
X₁₄ is Y;
X₁₅ is L; and
X₁₆ is H.
CDR-L2. X₁-X₂-X₃-X₄-X₅-X₆-X₇ (SEQ ID NO: 9), wherein;
X₁ is K;
X₂ is V;
X₃ is S;
X₄ is N;
X₅ is R;
X₆ is F; and
X₇ is R.
and
CDR-L3. X₁-X₂-X₃-X₄-X₅-X₆-X₇-X₈-X₉ (SEQ ID NO:10) wherein:
X₁ is S;
X₂ is Q;
X₃ is S;
X₄ is T;
X₅ is H;
X₆ is V;
X₇ is P;
X₈ is W; and
X₉ is T,
and 2) a human acceptor framework comprising at least one acceptor sequence selected from the group consisting of SEQ ID NO.:17, SEQ ID NO.:18, SEQ ID NO.:19, SEQ ID NO.:20, SEQ ID NO.:21, SEQ ID NO.:22, SEQ ID NO.:23 and SEQ ID NO.:24.

2. The binding protein according to claim 1, wherein said at least one CDR comprises an amino acid sequence selected from the group consisting of:
SEQ ID NO.:11, SEQ ID NO.:12, SEQ ID NO.:13, SEQ ID NO.:14, SEQ ID NO.:15, and SEQ ID NO.:16.

3. The binding protein according to claim 1, wherein said binding protein comprises at least 3 CDRs.

4. The binding protein according to claim 3, wherein said at least 3 CDRs are selected from a variable domain CDR set consisting of:

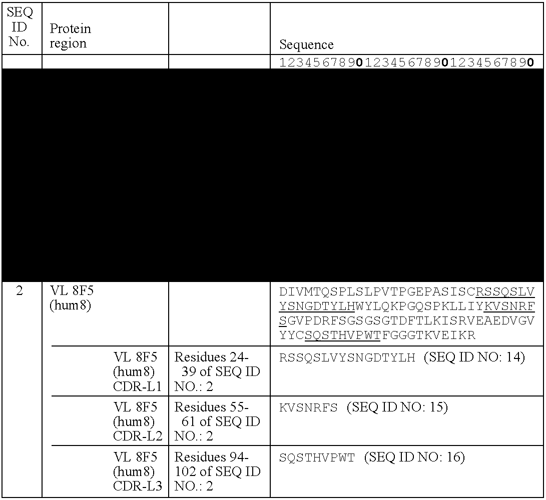

5. The binding protein according to claim 4, two variable domain CDR sets.

6. The binding protein according to claim 5, wherein said two variable domain CDR sets are VH 8F5 CDR Set & VL 8F5 CDR Set.

7. The binding protein according to claim 3, further comprising a human acceptor framework.

8. The binding protein according to claim 4, further comprising a human acceptor framework.

9. The binding protein according to claim 5, further comprising a human acceptor framework.

10. The binding protein according to claim 6, further comprising a human acceptor framework.

11. The binding protein according to claim 7, wherein said human acceptor framework comprises an amino acid sequence selected from the group consisting of:
SEQ ID NO.:17, SEQ ID NO.: 18, SEQ ID NO.:19, SEQ ID NO.:20, SEQ ID NO.:21, SEQ ID NO.:22, SEQ ID NO.:23 and SEQ ID NO.:24.

12. The binding protein according to claim 8, wherein said human acceptor framework comprises an amino acid sequence selected from the group consisting of:
SEQ ID NO.:17, SEQ ID NO.: 18, SEQ ID NO.:19, SEQ ID NO.:20, SEQ ID NO.:21, SEQ ID NO.:22, SEQ ID NO.:23 and SEQ ID NO.:24.

13. The binding protein according to claim 9, wherein said human acceptor framework comprises an amino acid sequence selected from the group consisting of:
SEQ ID NO.:17, SEQ ID NO.:18, SEQ ID NO.:19, SEQ ID NO.:20, SEQ ID NO.:21, SEQ ID NO.:22, SEQ ID NO.:23 and SEQ ID NO.:24.

14. The binding protein according to claim 10, wherein said human acceptor framework comprises amino acid sequence selected from the group consisting of:
SEQ ID NO.:17, SEQ ID NO.:18, SEQ ID NO.:19, SEQ ID NO.:20, SEQ ID NO.:21, SEQ ID NO.:22, SEQ ID NO.:23 and SEQ ID NO.:24.

15. The binding protein according to claim 1, wherein said binding protein comprises at least one variable domain having an amino acid sequence selected from the group consisting of: SEQ ID NO.:1 and SEQ ID NO.:2.

16. The binding protein according to claim 15 wherein said binding protein comprises two variable domains, wherein said two variable domains have amino acid sequences selected from the group consisting of: SEQ ID NO.:1 and SEQ ID NO.:2.

17. The binding protein according to claim 7, wherein said human acceptor framework comprises at least one Framework Region amino acid substitution at a key residue, said key residue selected from the group consisting of:
a residue adjacent to a CDR;
a glycosylation site residue;
a rare residue;
a residue capable of interacting with Aβ(1-42) globulomer;
a residue capable of interacting with a CDR;
a canonical residue;
a contact residue between heavy chain variable region and light chain variable region;
a residue within a Vernier zone; and
a residue in a region that overlaps between a Chothia-defined variable heavy chain CDR1 and a Kabat-defined first heavy chain framework.

18. The binding protein according to claim 10, wherein said human acceptor framework comprises at least one Framework Region amino acid substitution at a key residue, said key residue selected from the group consisting of:
a residue adjacent to a CDR;
a glycosylation site residue;
a rare residue;
a residue capable of interacting with an Aβ(1-42) globulomer;
a residue capable of interacting with a CDR;
a canonical residue;
a contact residue between heavy chain variable region and light chain variable region;
a residue within a Vernier zone; and
a residue in a region that overlaps between a Chothia-defined variable heavy chain CDR1 and a Kabat-defined first heavy chain framework.

19. The binding protein according to claim 16, wherein said human acceptor framework comprises at least one Framework Region amino acid substitution at a key residue, said key residue selected from the group consisting of:
a residue adjacent to a CDR;
a glycosylation site residue;
a rare residue;
a residue capable of interacting with an Aβ(1-42) globulomer;
a residue capable of interacting with a CDR;
a canonical residue;
a contact residue between heavy chain variable region and light chain variable region;
a residue within a Vernier zone; and
a residue in a region that overlaps between a Chothia-defined variable heavy chain CDR1 and a Kabat-defined first heavy chain framework.

20. The binding protein according to claim 7, wherein said human acceptor framework comprises at least one Framework Region amino acid substitution, wherein the amino acid sequence of the framework is at least 65% identical to the sequence of said human acceptor framework and comprises at least 52 amino acid residues identical to said human acceptor framework.

21. The binding protein according to claim 10, wherein said human acceptor framework comprises at least one Framework Region amino acid substitution, wherein the amino acid sequence of the framework is at least 65% identical to the sequence of said human acceptor framework and comprises at least 52 amino acid residues identical to said human acceptor framework.

22. The binding protein according to claim 16, wherein said human acceptor framework comprises at least one Framework Region amino acid substitution, wherein the amino acid sequence of the framework is at least 65% identical to the sequence of said human acceptor framework and comprises at least 52 amino acid residues identical to said human acceptor framework.

23. The binding protein according to claim 1, wherein said binding protein comprises at least one variable domain having an amino acid sequence selected from the group consisting of: SEQ ID NO.:1 and SEQ ID NO.:2 and said binding protein preferentially binds to the soluble form of Aβ(1-42) globulomer as compared to the fibrillar form.

24. The binding protein according to claim 23 wherein said binding protein comprises two variable domains, wherein said two variable domains have amino acid sequences of: SEQ ID NO.:1 and SEQ ID NO.:2.

25. The binding protein according to claim 20, wherein said binding protein comprises at least one variable domain having an amino acid sequence selected from the group consisting of: SEQ ID NO.:1 and SEQ ID NO.:2 and said binding protein preferentially binds to the soluble form of Aβ(1-42) globulomer as compared to the fibrillar form.

26. The binding protein according to claim 21, wherein said binding protein comprises at least one variable domain having an amino acid sequence selected from the group consisting of: SEQ ID NO.:1 and SEQ ID NO.:2.

27. The binding protein according to claim 22, wherein said binding protein comprises at least one variable domain having an amino acid sequence selected from the group consisting of: SEQ ID NO.:1 and SEQ ID NO.:2.

28. The binding protein according to claim 1, wherein the binding protein binds Aβ(1-42) globulomer and said binding protein preferentially binds to the soluble form of Aβ(1-42) globulomer as compared to the fibrillar form.

29. The binding protein according to claim 4, wherein the binding protein binds Aβ(1-42) globulomer and said binding protein preferentially binds to the soluble form of Aβ(1-42) globulomer as compared to the fibrillar form.

30. The binding protein according to claim 6, wherein the binding protein binds Aβ(1-42) globulomer and said binding protein preferentially binds to the soluble form of Aβ(1-42) globulomer as compared to the fibrillar form.

31. The binding protein according to claim 7, wherein the binding protein binds Aβ(1-42) globulomer and said binding protein preferentially binds to the soluble form of Aβ(1-42) globulomer as compared to the fibrillar form.

32. The binding protein according to claim 11, wherein the binding protein binds Aβ(1-42) globulomer and said binding protein preferentially binds to the soluble form of Aβ(1-42) globulomer as compared to the fibrillar form.

33. The binding protein according to claim 15, wherein the binding protein binds Aβ(1-42) globulomer and said binding protein preferentially binds to the soluble form of Aβ(1-42) globulomer as compared to the fibrillar form.

34. The binding protein according to claim 17, wherein the binding protein binds Aβ(1-42) globulomer and said binding protein preferentially binds to the soluble form of Aβ(1-42) globulomer as compared to the fibrillar form.

35. The binding protein according to claim 20, wherein the binding protein binds Aβ(1-42) globulomer and said binding protein preferentially binds to the soluble form of Aβ(1-42) globulomer as compared to the fibrillar form.

36. The binding protein according to claim 23, wherein the binding protein binds Aβ(1-42) globulomer and said binding protein preferentially binds to the soluble form of Aβ(1-42) globulomer as compared to the fibrillar form.

37. The binding protein according to claim 28, wherein the binding protein modulates a biological function of Aβ(1-42) globulomer.

38. The binding protein according to claim 33, wherein the binding protein modulates a biological function of Aβ(1-42) globulomer.

39. The binding protein according to claim 36, wherein the binding protein modulates a biological function of Aβ(1-42) globulomer.

40. The binding protein according to claim 28, wherein the binding protein neutralizes Aβ(1-42) globulomer.

41. The binding protein according to claim 33, wherein the binding protein neutralizes Aβ(1-42) globulomer.

42. The binding protein according to claim 36, wherein the binding protein neutralizes Aβ(1-42) globulomer.

43. The binding protein according to claim 28, wherein said binding protein has a dissociation constant (K_{D}) to said target selected from the group consisting of: at most about 10⁻⁷ M, at most about 10⁻⁸ M, at most about 10⁻⁹ M, at most about 10⁻¹⁰ M, at most about 10⁻¹¹ M, at most about 10⁻¹² M, and at most about 10⁻¹³ M.

44. The binding protein according to claim 33, wherein said binding protein has a dissociation constant (K_{D}) to said target selected from the group consisting of: at most about 10⁻⁷ M, at most about 10⁻⁸ M, at most about 10⁻⁹ M, at most about 10⁻¹⁰ M, at most about 10⁻¹¹ M, at most about 10⁻¹² M, and at most about 10⁻¹³ M.

45. The binding protein according to claim 35, wherein said binding protein has a dissociation constant (K_{D}) to said target selected from the group consisting of: at most about 10⁻⁷ M, at most about 10⁻⁸ M, at most about 10⁻⁹ M, at most about 10⁻¹⁰ M, at most about 10⁻¹¹ M, at most about 10⁻¹² M, and at most about 10⁻¹³ M.

46. The binding protein according to claim 36, wherein said binding protein has a dissociation constant (K_{D}) to said target selected from the group consisting of: at most about 10⁻⁷ M, at most about 10⁻⁸ M, at most about 10⁻⁹ M, at most about 10⁻¹⁰ M, at most about 10⁻¹¹ M, at most about 10⁻¹² M, and at most about 10⁻¹³ M.

47. An antibody construct comprising said binding protein of any one of claims 1-46, said antibody construct further comprising a linker polypeptide or an immunoglobulin constant domain.

48. The antibody construct according to claim 47, wherein said binding protein is selected from the group consisting of:
an immunoglobulin a humanized antibody,
molecule, a Fab,
a monoclonal antibody, a Fab′,
a chimeric antibody, a F(ab′)2,
a CDR-grafted antibody, a Fv,
a disulfide linked Fv, a mutated antibody, a
a scFv, dual variable domain
a single domain antibody, antibody
a diabody, and
a multispecific antibody, a bispecific antibody.
a dual specific antibody,
a isotype antibody,

49. The antibody construct according to claim 47, wherein said binding protein comprises a heavy chain immunoglobulin constant domain selected from the group consisting of:
a human IgM constant domain,
domain, a human IgG4 constant
a human IgG1 constant domain,
domain, a human IgE constant
a human IgG2 constant domain,
domain, and
a human IgG3 constant a human IgA constant
domain, domain.

50. The antibody construct according to claim 47, comprising an immunnoglobulin constant domain having an amino acid sequence selected from the group consisting of: SEQ ID NO.:25, SEQ ID NO.:26, SEQ ID NO.:27 and SEQ ID NO.:28.

51. An antibody conjugate comprising an antibody construct described in any one of claims 47-50, said antibody conjugate further comprising an agent selected from the group consisting of: an immunoadhension molecule, an imaging agent, a therapeutic agent, and a cytotoxic agent.

52. The antibody conjugate according to claim 51, wherein said agent is an imaging agent selected from the group consisting of a radiolabel, an enzyme, a fluorescent label, a luminescent label, a bioluminescent label, a magnetic label, and biotin.

53. The antibody conjugate according to claim 52, wherein said radiolabel is selected from the group consisting of: ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

54. The antibody conjugate according to claim 51, wherein said agent is a therapeutic or cytotoxic agent selected from the group consisting of: an anti-metabolite, an alkylating agent, an antibiotic, a growth factor, a cytokine, an anti-angiogenic agent, an anti-mitotic agent, an anthracycline, toxin, and an apoptotic agent.

55. The antibody construct according to claim 49, wherein said binding protein possesses a human glycosylation pattern.

56. The antibody conjugate according to claim 51, wherein said binding protein possesses a human glycosylation pattern.

57. The binding protein according to claim 3, wherein said binding protein exists as a crystal.

58. The antibody construct according to claim 47, wherein said antibody construct exists as a crystal.

59. The antibody conjugate according to claim 51, wherein said antibody construct exists as a crystal.

60. The binding protein according to claim 57, wherein said crystal is a carrier-free pharmaceutical controlled release crystal.

61. The antibody construct according to claim 58, wherein said crystal is a carrier-free pharmaceutical controlled release crystal.

62. The antibody conjugate according to claim 59, wherein said crystal is a carrier-free pharmaceutical controlled release crystal.

63. The binding protein according to claim 57, wherein said binding protein has a greater half life in vivo than the soluble counterpart of said binding protein.

64. The antibody construct according to claim 58, wherein said antibody construct has a greater half life in vivo than the soluble counterpart of said antibody construct.

65. The antibody conjugate according to claim 59, wherein said antibody conjugate has a greater half life in vivo than the soluble counterpart of said antibody conjugate.

66. The binding protein according to claim 57, wherein said binding protein retains biological activity.

67. The antibody construct according to claim 58, wherein said antibody construct retains biological activity.

68. The antibody conjugate according to claim 59, wherein said antibody conjugate retains biological activity.

69. An isolated nucleic acid molecule encoding a binding protein, wherein the amino acid sequence of said variable heavy chain of said binding protein has at least 70% identity to SEQ ID NO:1.

70. An isolated nucleic acid molecule encoding a binding protein, wherein the amino acid sequence of said variable light chain of said binding protein has at least 70% identity to SEQ ID NO:2.

71. An isolated nucleic acid molecule encoding a binding protein amino acid sequence of any one of claims 1-46.

72. An isolated nucleic acid molecule encoding an antibody contruct amino acid sequence of any one of claims 47-50.

73. An isolated nucleic acid molecule encoding an antibody conjugate amino acid sequence of any one of claims 51-53.

74. A vector comprising said nucleic acid molecule of any one of claims 71-73.

75. A host cell comprising said vector of claim 74.

76. The host cell of claim 75, wherein said host cell is a prokaryotic cell.

77. The host cell according to claim 76, wherein said host cell is Escherichia coli.

78. The host cell according to claim 75, wherein said host cell is a eukaryotic cell.

79. The host cell according to claim 78, wherein said eukaryotic cell is selected from the group consisting of protist cell, animal cell, plant cell and fungal cell.

80. The host cell according to claim 79, wherein said animal cell is selected from the group consisting of a mammalian cell, an avian cell, and an insect cell.

81. The host cell according to claim 80, wherein said mammalian cell is a CHO cell.

82. The host cell according to claim 80, wherein said host cell is COS.

83. The host cell according to claim 80, wherein said fungal cell is a yeast cell.

84. The host cell according to claim 83, wherein said yeast cell is Saccharomyces cerevisiae.

85. The host cell according to claim 80, wherein said insect cell is Sf9.

86. A method of producing a protein capable of binding Aβ(1-42) globulomer, comprising culturing a host cell of any one of claims 75-85 for a time and under conditions sufficient to produce a binding protein capable of binding Aβ(1-42) globulomer.

87. An isolated protein produced according to the method of claim 86.

88. A composition for the release of a binding protein said composition comprising:
(a) a formulation, wherein said formulation comprises a crystallized binding protein, according to any one of claims 57-68, and an ingredient; and
(b) at least one polymeric carrier.

89. The composition according to claim 88, wherein said polymeric carrier is at least one polymer selected the group consisting of: poly (acrylic acid), poly (cyanoacrylates), poly (amino acids), poly (anhydrides), poly (depsipeptide), poly (esters), poly (lactic acid), poly (lactic-co-glycolic acid) or PLGA, poly (b-hydroxybutryate), poly (caprolactone), poly (dioxanone); poly (ethylene glycol), poly ((hydroxypropyl) methacrylamide, poly [(organo) phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polyeaccharides, blends and copolymers thereof.

90. The composition according to claim 87, wherein said ingredient is selected from the group consisting of albumin, sucrose, trehalose, lactitol, gelatin, hydroxypropyl-cyclodextrin, methoxypolyethylene glycol and polyethylene glycol.

91. A method for treating a mammal suspected of having an amyloidosis comprising administering to the mammal said composition of claim 88 in an amount sufficient to effect said treatment.

92. A pharmaceutical composition comprising the binding protein of claim 1, and a pharmaceutically acceptable carrier.

93. The pharmaceutical composition of claim 91 wherein said pharmaceutically acceptable carrier functions as adjuvant useful to increase the absorption, or dispersion of said binding protein.

94. The pharmaceutical composition of claim 92 wherein said adjuvant is hyaluronidase.

95. The pharmaceutical composition of claim 91 further comprising at least one additional therapeutic agent for treating a disorder in which presence of Aβ(1-42) globulomer is detrimental.

96. The method of claim 95 wherein said at least one additional therapeutic agent is selected from the group consisting of a cholesterinase inhibitor, a TNF antagonist, a cytokine antagonist, a partial NMDA receptor blocker, a glycosaminoglycan mimetic, an inhibitor or allosteric modulator of gamma secretase, a luteinizing hormone blockade gonadotropin releasing hormone agonist, a serotinin 5-HT1A receptor antagonist, a chelating agent, a neuronal selective L-type calcium channel blocker, an immunomodulator, an amyloid fibrillogenesis inhibitor or amyloid protein deposition inhibitor, a PDE4 inhibitor, a histamine agonist, a receptor protein for advanced glycation end products, a PARP stimulator, a serotonin 6-receptor antagonist, a 5-HT4 receptor agonist, a human steroid, a glucose uptake stimulant which enhanceds neuronal metabolism, a selective CB1 antagonist, a partial agonist at benzodiazepine receptors, an amyloid beta production antagonist or inhibitor, an amyloid beta deposition inhibitor, a NNR alpha-7 partial antagonist, a therapeutic targeting PDE4, a RNA translation inhibitor, a muscarinic agonist, a nerve growth factor receptor agonist, a NGF receptor agonist and a gene therapy modulator.

97. A method for reducing Aβ(1-42) globulomer activity comprising contacting Aβ(1-42) globulomer with the binding protein of claim 1 such that Aβ(1-42) globulomer activity is reduced.

98. A method for reducing human Aβ(1-42) globulomer activity in a human subject suffering from a disorder in which Aβ(1-42) globulomer is detrimental, comprising administering to the human subject the binding protein of claim 1 such that human Aβ(1-42) globulomer activity in the human subject is reduced.

99. A method for treating a subject for a disease or a disorder in which Aβ(1-42) globulomer activity is detrimental by administering to the subject the binding protein of claim 1 in an amount sufficient to effect said treatment.

100. The method of claim 99, wherein said disease or disorder is selected from the group consisting of Alpha1-antitrypsin-deficiency, C1-inhibitor deficiency angioedema, Antithrombin deficiency thromboembolic disease, Kuru, Creutzfeld-Jacob disease/scrapie, Bovine spongiform encephalopathy, Gerstmann-Straussler-Scheinker disease, Fatal familial insomnia, Huntington's disease, Spinocerebellar ataxia, Machado-Joseph atrophy, Dentato-rubro-pallidoluysian atrophy, Frontotemporal dementia, Sickle cell anemia, Unstable hemoglobin inclusion-body hemolysis, Drug-induced inclusion body hemolysis, Parkinson's disease, Systemic AL amyloidosis, Nodular AL amyloidosis, Systemic AA amyloidosis, Prostatic amyloid, Hemodialysis amyloidosis, Hereditary (Icelandic) cerebral angiopathy, Huntington's disease, Familial visceral amyloid, Familial visceral polyneuropathy, Familial visceral amyloidosis, Senile systemic amyloidosis, Familial amyloid neurophathy, Familial cardiac amyloid, Alzheimer's disease, Down's syndrome, Medullary carcinoma thyroid and Type 2 diabetes mellitus (T2DM).

101. A method of treating a patient suffering from a disorder in which Aβ(1-42) globulomer is detrimental comprising the step of administering the binding protein of claim 1 before, concurrent, or after the administration of at least one second agent, wherein said at least one second agent is selected from the group consisting of a cholesterinase inhibitor, a partial NMDA receptor blocker, a glycosaminoglycan mimetic, a TNF antagonist, a cytokine antagonist, an inhibitor or allosteric modulator of gamma secretase, a luteinizing hormone blockade gonadotropin releasing hormone agonist, a serotinin 5-HT1A receptor antagonist, a chelating agent, a neuronal selective L-type calcium channel blocker, an immunomodulator, an amyloid fibrillogenesis inhibitor or amyloid protein deposition inhibitor, a PDE4 inhibitor, a histamine agonist, a receptor protein for advanced glycation end products, a PARP stimulator, a serotonin 6-receptor antagonist, a 5-HT4 receptor agonist, a human steroid, a glucose uptake stimulant which enhances neuronal metabolism, a selective CB1 antagonist, a partial agonist at benzodiazepine receptors, an amyloid beta production antagonist or inhibitor, an amyloid beta deposition inhibitor, a NNR alpha-7 partial antagonist, a therapeutic targeting PDE4, a RNA translation inhibitor, a muscarinic agonist, a nerve growth factor receptor agonist, a NGF receptor agonist and a gene therapy modulator.

102. The method of claim 101 wherein said cholesterinase inhibitor is selected from the group consisting of Tacrine, Donepezil, Rivastigmine and Galantamine.

103. The method of claim 101 wherein said partial NMDA receptor blocker is Memantine.

104. The method according to claim 98, wherein said administering to the subject is by at least one mode selected from the group consisting of parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

105. A method of diagnosing Alzheimer's Disease in a patient suspected of having this disease comprising the steps
of:
a. isolating a biological sample from said patient;
b. contacting said biological sample with said binding protein of claim 1 for a time and under conditions sufficient for formation of globulomer/binding protein complexes; and
c. detecting presence of said globulomer/binding protein complexes in said sample, presence of said complexes indicating a diagnosis of Alzheimer's Disease in said patient.

106. A method of diagnosing Alzheimer's Disease in a patient suspected of having this disease comprising the steps
of:
a. isolating a biological sample from said patient;
b. contacting said biological sample with said binding protein of claim 1 for a time and under conditions sufficient for the formation of globulomer/binding protein complexes;
c. adding a conjugate to the resulting globulomer/binding protein complexes for a time and under conditions sufficient to allow said conjugate to bind to the bound binding protein, wherein said conjugate comprises an anti-binding protein antibody attached to a signal generating compound capable of generating a detectable signal; and
d. detecting the presence of said binding protein which may be present in said biological sample by detecting a signal generated by said signal generating compound, said signal indicating a diagnosis of Alzheimer's Disease in said patient.


SEQUENCE LISTING (112 sequences)

SEQ ID NO: 1 - protein, 112 aa, Homo sapiens
EVQLVESGGGLVQPGGSLRLSCAASGFTFSSYGMSWVRQAPGKGLELVASINSNGGSTYYPDSVKGRFTISRDNAKNTLYLQMNSLRAEDTAVYYCASGDYWGQGTLVTVSS

SEQ ID NO: 2 - protein, 113 aa, Homo sapiens
DIVMTQSPLSLPVTPGEPASISCRSSQSLVYSNGDTYLHWYLQKPGQSPKLLIYKVSNRFSGVPDRFSGSGSGTDFTLKISRVEAEDVGVYYCSQSTHVPWTFGGGTKVEIKR

SEQ ID NO: 3 - DNA, 336 bp, Homo sapiens
gaggtgcagctggtggagtctgggggaggcttagtgcagcctggagggtccctgagactctcctgtgcagcctctggattcactttcagtagctatggcatgtcttgggttcgccaggctccaggcaaggggctggaattggtcgcaagcatcaatagtaatggtggtagcacctattatccagacagtgtgaagggccgattcaccatctccagagacaatgccaagaacaccctgtacctgcaaatgaacagtctgagggctgaggacacagccgtgtattactgtgcaagtggtgactactggggccaaggcacccttgtcacagtctcctca

SEQ ID NO: 4 - DNA, 339 bp, Homo sapiens
gatattgtgatgacccaatctccactctccctgcctgtcactcctggagaaccagcctccatctcttgcagatctagtcagagccttgtatatagtaatggagacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatgtgggagtttattactgctctcaaagtacacatgttccttggacgttcggtggaggcaccaaggtagaaatcaaacgg

SEQ ID NO: 5 - protein, 5 aa, Artificial Sequence
  Description of Artificial Sequence Synthetic peptide
SYGMS

SEQ ID NO: 6 - protein, 17 aa, Artificial Sequence
  Description of Artificial Sequence Synthetic peptide
SINSNGGSTYYPDSVKG

SEQ ID NO: 7 - protein, 4 aa, Artificial Sequence
  Description of Artificial Sequence Synthetic peptide
SGDY

SEQ ID NO: 8 - protein, 16 aa, Artificial Sequence
  Description of Artificial Sequence Synthetic peptide
RSSQSLVYSNGDTYLH

SEQ ID NO: 9 - protein, 7 aa, Artificial Sequence
  Description of Artificial Sequence Synthetic peptide
KVSNRFS

SEQ ID NO: 10 - protein, 9 aa, Artificial Sequence
  Description of Artificial Sequence Synthetic peptide
SQSTHVPWT

SEQ ID NO: 11 - protein, 5 aa, Homo sapiens
SYGMS

SEQ ID NO: 12 - protein, 17 aa, Homo sapiens
SINSNGGSTYYPDSVKG

SEQ ID NO: 13 - protein, 4 aa, Homo sapiens
SGDY

SEQ ID NO: 14 - protein, 16 aa, Homo sapiens
RSSQSLVYSNGDTYLH

SEQ ID NO: 15 - protein, 7 aa, Homo sapiens
KVSNRFS

SEQ ID NO: 16 - protein, 9 aa, Homo sapiens
SQSTHVPWT

SEQ ID NO: 17 - protein, 30 aa, Homo sapiens
EVQLLESGGGLVQPGGSLRLSCAASGFTFS

SEQ ID NO: 18 - protein, 14 aa, Homo sapiens
WVRQAPGKGLEWVS

SEQ ID NO: 19 - protein, 31 aa, Homo sapiens
RFTISRDNSKNTLYLQMNSLRAEDTAVYYCA

SEQ ID NO: 20 - protein, 11 aa, Homo sapiens
WGQGTLVTVSS

SEQ ID NO: 21 - protein, 23 aa, Homo sapiens
DIVMTQSPLSLPVTPGEPASISC

SEQ ID NO: 22 - protein, 15 aa, Homo sapiens
WYLQKPGQSPQLLIY

SEQ ID NO: 23 - protein, 32 aa, Homo sapiens
GVPDRFSGSGSGTDFTLKISRVEAEDVGVYYC

SEQ ID NO: 24 - protein, 11 aa, Homo sapiens
FGGGTKVEIKR

SEQ ID NO: 25 - protein, 330 aa, Homo sapiens
ASTKGPSVFFLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 105 aa, Homo sapiens
TVAAPSVFIFPPSDEQLKSGTASVVCLLNNFYPREAKVQWKVDNALQSGNSQESVTEQDSKSTYSLSSTLTLSKADYEKHKVYACEVTHQGLSSPVTKSFNRGEC

SEQ ID NO: 28 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 42 aa, Homo sapiens [sequence omitted]

SEQ ID NO: 30 - protein, 40 aa, Homo sapiens
DAEFRHDSGYEVHHQKLVFFAEDVGSNKGAIIGLMVGGVV

SEQ ID NO: 31 - DNA, 41 bp, Artificial Sequence
  Description of Artificial Sequence Synthetic primer
ctggcaagggcctggagctggtggccagcatcaacagcaac

SEQ ID NO: 32 - DNA, 41 bp, Artificial Sequence
  Description of Artificial Sequence Synthetic primer
gttgctgttgatgctggccaccagctccaggcccttgccag

SEQ ID NO: 33 - DNA, 27 bp, Artificial Sequence
  Description of Artificial Sequence Synthetic primer
ccatcccgggaggagatgaccaagaac

SEQ ID NO: 34 - DNA, 27 bp, Artificial Sequence
  Description of Artificial Sequence Synthetic primer
gttcttggtcatctcctcccgggatgg

SEQ ID NO: 35 - DNA, 18 bp, Artificial Sequence
  Description of Artificial Sequence Synthetic primer
ccacatggacagaggccg

SEQ ID NO: 36 - DNA, 17 bp, Artificial Sequence
  Description of Artificial Sequence Synthetic primer
agggcagcgctgggtgc

SEQ ID NO: 37 - DNA, 48 bp, Artificial Sequence
  Description of Artificial Sequence Synthetic primer
catctcttcctcagcacctgaagccgcggggggaccgtcagtcttcct

SEQ ID NO: 38 - DNA, 48 bp, Artificial Sequence
  Description of Artificial Sequence Synthetic primer
aggaagactgacggtccccccgcggcttcaggtgctgaggaagagatg

SEQ ID NO: 39 - DNA, 38 bp, Artificial Sequence
  Description of Artificial Sequence Synthetic oligonucleotide
agacgctgttgccttatacgcgtccaccatggagttcg

SEQ ID NO: 40 - DNA, 33 bp, Artificial Sequence
  Description of Artificial Sequence Synthetic oligonucleotide
acagccagctcaggccgaactccatggtggacg

SEQ ID NO: 41 - DNA, 30 bp, Artificial Sequence
  Description of Artificial Sequence Synthetic oligonucleotide
gcctgagctggctgttcctggtggccatcc

SEQ ID NO: 42 - DNA, 30 bp, Artificial Sequence
  Description of Artificial Sequence Synthetic oligonucleotide
actgcacgcccttcaggatggccaccagga

SEQ ID NO: 43 - DNA, 30 bp, Artificial Sequence
  Description of Artificial Sequence Synthetic oligonucleotide
tgaagggcgtgcagtgcgaggtgcagctgg

SEQ ID NO: 44 - DNA, 29 bp, Artificial Sequence
  Description of Artificial Sequence Synthetic oligonucleotide
gccgccgctctccaccagctgcacctcgc

SEQ ID NO: 45 - DNA, 28 bp, Artificial Sequence
  Description of Artificial Sequence Synthetic oligonucleotide
tggagagcggcggcggcctggtgcagcc

SEQ ID NO: 46 - DNA, 27 bp, Artificial Sequence
  Description of Artificial Sequence Synthetic oligonucleotide
caggctgccgccaggctgcaccaggcc

SEQ ID NO: 47 - DNA, 27 bp, Artificial Sequence
  Description of Artificial Sequence Synthetic oligonucleotide
tggcggcagcctgcgcctgagctgcgc

SEQ ID NO: 48 - DNA, 28 bp, Artificial Sequence
  Description of Artificial Sequence Synthetic oligonucleotide
tgaagccgctggcggcgcagctcaggcg

SEQ ID NO: 49 - DNA, 31 bp, Artificial Sequence
  Description of Artificial Sequence Synthetic oligonucleotide
cgccagcggcttcaccttcagcagctacggc

SEQ ID NO: 50 - DNA, 32 bp, Artificial Sequence
  Description of Artificial Sequence Synthetic oligonucleotide
gcgcacccagctcatgccgtagctgctgaagg

SEQ ID NO: 51 - DNA, 28 bp, Artificial Sequence
  Description of Artificial Sequence Synthetic oligonucleotide
atgagctgggtgcgccaggcccctggca

SEQ ID NO: 52 - DNA, 28 bp, Artificial Sequence
  Description of Artificial Sequence Synthetic oligonucleotide
cccactccaggcccttgccaggggcctg

SEQ ID NO: 53 - DNA, 32 bp, Artificial Sequence
  Description of Artificial Sequence Synthetic oligonucleotide
agggcctggagtgggtggccagcatcaacagc

SEQ ID NO: 54 - DNA, 30 bp, Artificial Sequence
  Description of Artificial Sequence Synthetic oligonucleotide
tgctgccgccgttgctgttgatgctggcca

SEQ ID NO: 55 - DNA, 31 bp, Artificial Sequence
  Description of Artificial Sequence Synthetic oligonucleotide
aacggcggcagcacctactaccctgacagcg

SEQ ID NO: 56 - DNA, 33 bp, Artificial Sequence
  Description of Artificial Sequence Synthetic oligonucleotide
tgaagcggcccttcacgctgtcagggtagtagg

SEQ ID NO: 57 - DNA, 30 bp, Artificial Sequence
  Description of Artificial Sequence Synthetic oligonucleotide
tgaagggccgcttcaccatcagccgcgaca

SEQ ID NO: 58 - DNA, 32 bp, Artificial Sequence
  Description of Artificial Sequence Synthetic oligonucleotide
cagggtgttcttggcgttgtcgcggctgatgg

SEQ ID NO: 59 - DNA, 37 bp, Artificial Sequence
  Description of Artificial Sequence Synthetic oligonucleotide
acgccaagaacaccctgtacctgcagatgaacagcct

SEQ ID NO: 60 - DNA, 34 bp, Artificial Sequence
  Description of Artificial Sequence Synthetic oligonucleotide
tgtcctcggcgcgcaggctgttcatctgcaggta

SEQ ID NO: 61 - DNA, 32 bp, Artificial Sequence
  Description of Artificial Sequence Synthetic oligonucleotide
gcgcgccgaggacaccgccgtgtactactgcg

SEQ ID NO: 62 - DNA, 33 bp, Artificial Sequence
  Description of Artificial Sequence Synthetic oligonucleotide
agtagtcgccgctggcgcagtagtacacggcgg

SEQ ID NO: 63 - DNA, 29 bp, Artificial Sequence
  Description of Artificial Sequence Synthetic oligonucleotide
ccagcggcgactactggggccagggcacc

SEQ ID NO: 64 - DNA, 32 bp, Artificial Sequence
  Description of Artificial Sequence Synthetic oligonucleotide
tgaggagacggtgacgagggtgccctggcccc

SEQ ID NO: 65 - DNA, 37 bp, Artificial Sequence
  Description of Artificial Sequence Synthetic oligonucleotide
ctcgtcaccgtctcctcaggtgagtcctcacaacctc

SEQ ID NO: 66 - DNA, 40 bp, Artificial Sequence
  Description of Artificial Sequence Synthetic oligonucleotide
gcgtcacggggtaaatatctagaggttgtgaggactcacc

SEQ ID NO: 67 - DNA, 20 bp, Artificial Sequence
  Description of Artificial Sequence Synthetic oligonucleotide
agacgctgttgccttatacg

SEQ ID NO: 68 - DNA, 21 bp, Artificial Sequence
  Description of Artificial Sequence Synthetic oligonucleotide
gcgtcacggggtaaatatcta

SEQ ID NO: 69 - DNA, 21 bp, Artificial Sequence
  Description of Artificial Sequence Synthetic oligonucleotide
gcgtatatcccggttgttgct

SEQ ID NO: 70 - DNA, 39 bp, Artificial Sequence
  Description of Artificial Sequence Synthetic oligonucleotide
agcaacaaccgggatatacgcgtccaccatggacatgcg

SEQ ID NO: 71 - DNA, 32 bp, Artificial Sequence
  Description of Artificial Sequence Synthetic oligonucleotide
gctgggcaggcacgcgcatgtccatggtggac

SEQ ID NO: 72 - DNA, 28 bp, Artificial Sequence
  Description of Artificial Sequence Synthetic oligonucleotide
cgtgcctgcccagctgctgggcctgctg

SEQ ID NO: 73 - DNA, 31 bp, Artificial Sequence
  Description of Artificial Sequence Synthetic oligonucleotide
ccagggaaccacagcagcagcaggcccagca

SEQ ID NO: 74 - DNA, 31 bp, Artificial Sequence
  Description of Artificial Sequence Synthetic oligonucleotide
ctgctgtggttccctggcagccgctgcgaca

SEQ ID NO: 75 - DNA, 31 bp, Artificial Sequence
  Description of Artificial Sequence Synthetic oligonucleotide
gctctgggtcatcacgatgtcgcagcggctg

SEQ ID NO: 76 - DNA, 33 bp, Artificial Sequence
  Description of Artificial Sequence Synthetic oligonucleotide
tcgtgatgacccagagccctctgagcctgcctg

SEQ ID NO: 77 - DNA, 31 bp, Artificial Sequence
  Description of Artificial Sequence Synthetic oligonucleotide
gctcgccaggggtcacaggcaggctcagagg

SEQ ID NO: 78 - DNA, 32 bp, Artificial Sequence
  Description of Artificial Sequence Synthetic oligonucleotide
tgacccctggcgagcctgccagcatcagctgc

SEQ ID NO: 79 - DNA, 32 bp, Artificial Sequence
  Description of Artificial Sequence Synthetic oligonucleotide
gctctggctgctgcggcagctgatgctggcag

SEQ ID NO: 80 - DNA, 33 bp, Artificial Sequence
  Description of Artificial Sequence Synthetic oligonucleotide
cgcagcagccagagcctggtgtacagcaacggc

SEQ ID NO: 81 - DNA, 36 bp, Artificial Sequence
  Description of Artificial Sequence Synthetic oligonucleotide
ccagtgcaggtaggtgtcgccgttgctgtacaccag

SEQ ID NO: 82 - DNA, 35 bp, Artificial Sequence
  Description of Artificial Sequence Synthetic oligonucleotide
gacacctacctgcactggtacctgcagaagcctgg

SEQ ID NO: 83 - DNA, 34 bp, Artificial Sequence
  Description of Artificial Sequence Synthetic oligonucleotide
gcagcttagggctctggccaggcttctgcaggta

SEQ ID NO: 84 - DNA, 39 bp, Artificial Sequence
  Description of Artificial Sequence Synthetic oligonucleotide
ccagagccctaagctgctgatctacaaagtgagcaaccg

SEQ ID NO: 85 - DNA, 37 bp, Artificial Sequence
  Description of Artificial Sequence Synthetic oligonucleotide
ggcacgccgctgaagcggttgctcactttgtagatca

SEQ ID NO: 86 - DNA, 30 bp, Artificial Sequence
  Description of Artificial Sequence Synthetic oligonucleotide
cttcagcggcgtgcctgaccgcttcagcgg

SEQ ID NO: 87 - DNA, 29 bp, Artificial Sequence
  Description of Artificial Sequence Synthetic oligonucleotide
tgccgctgccgctgccgctgaagcggtca

SEQ ID NO: 88 - DNA, 32 bp, Artificial Sequence
  Description of Artificial Sequence Synthetic oligonucleotide
cagcggcagcggcaccgacttcaccctgaaga

SEQ ID NO: 89 - DNA, 32 bp, Artificial Sequence
  Description of Artificial Sequence Synthetic oligonucleotide
ctccacgcggctgatcttcagggtgaagtcgg

SEQ ID NO: 90 - DNA, 28 bp, Artificial Sequence
  Description of Artificial Sequence Synthetic oligonucleotide
tcagccgcgtggaggccgaggacgtggg

SEQ ID NO: 91 - DNA, 32 bp, Artificial Sequence
  Description of Artificial Sequence Synthetic oligonucleotide
tggctgcagtagtacacgcccacgtcctcggc

SEQ ID NO: 92 - DNA, 33 bp, Artificial Sequence
  Description of Artificial Sequence Synthetic oligonucleotide
cgtgtactactgcagccagagcacccacgtgcc

SEQ ID NO: 93 - DNA, 30 bp, Artificial Sequence
  Description of Artificial Sequence Synthetic oligonucleotide
ccgccgaaggtccaaggcacgtgggtgctc

SEQ ID NO: 94 - DNA, 32 bp, Artificial Sequence
  Description of Artificial Sequence Synthetic oligonucleotide
ttggaccttcggcggcggcaccaaagtggaga

SEQ ID NO: 95 - DNA, 39 bp, Artificial Sequence
  Description of Artificial Sequence Synthetic oligonucleotide
aggaaagtgcacttacgtttgatctccactttggtgccg

SEQ ID NO: 96 - DNA, 43 bp, Artificial Sequence
  Description of Artificial Sequence Synthetic oligonucleotide
tcaaacgtaagtgcactttcctaatctagatattcggctcgac

SEQ ID NO: 97 - DNA, 22 bp, Artificial Sequence
  Description of Artificial Sequence Synthetic oligonucleotide
cgtcgagccgaatatctagatt

SEQ ID NO: 98 - DNA, 21 bp, Artificial Sequence
  Description of Artificial Sequence Synthetic oligonucleotide
agcaacaaccgggatatacgc

SEQ ID NO: 99 - DNA, 22 bp, Artificial Sequence
  Description of Artificial Sequence Synthetic oligonucleotide
cgtcgagccgaatatctagatt

SEQ ID NO: 100 - protein, 112 aa, Homo sapiens
EVQLVESGGGLVQPGGSLKLSCAASGFTFSSYGMSWVRQTPDKRLELVASINSNGGSTYYPDSVKGRFTISRDNAKNTLYLQMSSLKSEDTAMYYCASGDYWGQGSTLTVSS

SEQ ID NO: 101 - protein, 112 aa, Homo sapiens
EVQLVESGGGLVQPGGSLRLSCAASGFTFSSYGMSWVRQAPGKGLEWVASINSNGGSTYYPDSVKGRFTISRDNAKNTLYLQMNSLRAEDTAVYYCASGDYWGQGTLVTVSS

SEQ ID NO: 102 - protein, 112 aa, Homo sapiens; identical to SEQ ID NO: 1

SEQ ID NO: 103 - protein, 87 aa, Homo sapiens
EVQLVESGGGLVKPGGSLRLSCAASGFTFSWVRQAPGKGLEWVSRFTISRDNAKNSLYLQMNSLRAEDTAVYYCARWGRGTLVTVSS

SEQ ID NO: 104 - protein, 112 aa, Homo sapiens
DVVMTQTPLSLPVSLGDQASISCRSSQSLVYSNGDTYLHWYLQKPGQSPKLLIYKVSNRFSGVPDRFSGSGSGTDFTLKISRVEAEDLGVYFCSQSTHVPWTFGGGTKLEIK

SEQ ID NO: 105 - protein, 112 aa, Homo sapiens
DIVMTQSPLSLPVTPGEPASISCRSSQSLVYSNGDTYLHWYLQKPGQSPKLLIYKVSNRFSGVPDRFSGSGSGTDFTLKISRVEAEDVGVYYCSQSTHVPWTFGGGTKVEIK

SEQ ID NO: 106 - protein, 80 aa, Homo sapiens
ELVMTQSPLSLPVTPGEPASISCWYLQKPGQSPQLLIYGVPDRFSGSGSGTDFTLKISRVEAEDVGVYYCFGGGTKVEIK

SEQ ID NO: 107 - DNA, 427 bp, Homo sapiens
  CDS (12)..(404)
acgcgtccaccatggagttcggcctgagctggctgttcctggtggccatcctgaagggcgtgcagtgcgaggtgcagctggtggagagcggcggcggcctggtgcagcctggcggcagcctgcgcctgagctgcgccgccagcggcttcaccttcagcagctacggcatgagctgggtgcgccaggcccctggcaagggcctggagtgggtggccagcatcaacagcaacggcggcagcacctactaccctgacagcgtgaagggccgcttcaccatcagccgcgacaacgccaagaacaccctgtacctgcagatgaacagcctgcgcgccgaggacaccgccgtgtactactgcgccagcggcgactactggggccagggcaccctcgtcaccgtctcctcaggtgagtcctcacaacctctaga

SEQ ID NO: 108 - protein, 131 aa, Homo sapiens
MEFGLSWLFLVAILKGVQCEVQLVESGGGLVQPGGSLRLSCAASGFTFSSYGMSWVRQAPGKGLEWVASINSNGGSTYYPDSVKGRFTISRDNAKNTLYLQMNSLRAEDTAVYYCASGDYWGQGTLVTVSS

SEQ ID NO: 109 - DNA, 427 bp, Homo sapiens
  CDS (12)..(404)
acgcgtccaccatggagttcggcctgagctggctgttcctggtggccatcctgaagggcgtgcagtgcgaggtgcagctggtggagagcggcggcggcctggtgcagcctggcggcagcctgcgcctgagctgcgccgccagcggcttcaccttcagcagctacggcatgagctgggtgcgccaggcccctggcaagggcctggagctggtggccagcatcaacagcaacggcggcagcacctactaccctgacagcgtgaagggccgcttcaccatcagccgcgacaacgccaagaacaccctgtacctgcagatgaacagcctgcgcgccgaggacaccgccgtgtactactgcgccagcggcgactactggggccagggcaccctcgtcaccgtctcctcaggtgagtcctcacaacctctaga

SEQ ID NO: 110 - protein, 131 aa, Homo sapiens
MEFGLSWLFLVAILKGVQCEVQLVESGGGLVQPGGSLRLSCAASGFTFSSYGMSWVRQAPGKGLELVASINSNGGSTYYPDSVKGRFTISRDNAKNTLYLQMNSLRAEDTAVYYCASGDYWGQGTLVTVSS

SEQ ID NO: 111 - DNA, 438 bp, Homo sapiens
  CDS (12)..(413)
acgcgtccaccatggacatgcgcgtgcctgcccagctgctgggcctgctgctgctgtggttccctggcagccgctgcgacatcgtgatgacccagagccctctgagcctgcctgtgacccctggcgagcctgccagcatcagctgccgcagcagccagagcctggtgtacagcaacggcgacacctacctgcactggtacctgcagaagcctggccagagccctaagctgctgatctacaaagtgagcaaccgcttcagcggcgtgcctgaccgcttcagcggcagcggcagcggcaccgacttcaccctgaagatcagccgcgtggaggccgaggacgtgggcgtgtactactgcagccagagcacccacgtgccttggaccttcggcggcggcaccaaagtggagatcaaacgtaagtgcactttcctaatctaga

SEQ ID NO: 112 - protein, 134 aa, Homo sapiens
MDMRVPAQLLGLLLLWFPGSRCDIVMTQSPLSLPVTPGEPASISCRSSQSLVYSNGDTYLHWYLQKPGQSPKLLIYKVSNRFSGVPDRFSGSGSGTDFTLKISRVEAEDVGVYYCSQSTHVPWTFGGGTKVEIK